(19) United States
(12) Patent Application Publication
(10) Pub. No.: US 2021/0169928 A1
(43) Pub. Date: Jun. 10, 2021

(54) Topical Exosome Compositions and Associated Methods

(71) Applicant: EriVan Bio, LLC, Alachua, FL (US)

(72) Inventors: Nasrin Nassiri Koopaei, Tehran (IR); Jeffrey L. Tate, Newberry, FL (US)

(21) Appl. No.: 16/936,090

(22) Filed: Jul. 22, 2020

Related U.S. Application Data
(60) Provisional application No. 62/877,155, filed on Jul. 22, 2019.

(51) Int. Cl. A61K 35/15; A61K 31/506; A61K 31/721; A61K 45/06

(52) U.S. Cl. CPC A61K 35/15; A61K 45/06; A61K 31/721; A61K 31/506

(57) ABSTRACT

Topical exosome compositions for treating a variety of conditions in a subject, including methods of making and using the same. Exemplary embodiments employ MSC exosomes and secretome as components of the compositions, including but not limited to A-MSC exosomes and secretome.

### CROSS REFERENCE TO RELATED APPLICATION

This application claims the benefit of, and priority to, U.S. Provisional Patent Application Ser. No. 62/877,155, filed Jul. 22, 2019, the entirety of which is incorporated by reference and commonly owned.

### FIELD OF THE INVENTION

The present disclosure relates generally to topical exosome compositions for use in treating a variety of conditions in a subject, as well as methods of making and using the same.

### BACKGROUND

Extracellular vesicles (EVs) including exosomes, microvesicles, and apoptotic bodies are nanoscale spherical vesicles that are released when the multivesicular bodies (MVBs) fuse with the cell plasma membrane. Consequently, they bud and are shed into the extracellular space and bodily fluids. Exosomes are increasingly important in cell biology because of their role in cell-to-cell communication and transfer of cellular materials, such as miRNA, lncRNA, mRNA, proteins, and DNA. This mechanism implies that exosomes underlie some illnesses such as cancer, neurological, cardiovascular, and rheumatologic diseases. Different cell types grown in vitro constantly and constitutively secrete exosomes into the culture media. Therefore, the conditioned culture medium of cells serves as the very food for exosome manufacturing. Notably, many biopharmaceutical companies are already producing the exosomes during the cell culture processes, but these exosomes are discarded as a waste by-product. Exosomes, however, can be applied in different translational paradigms comprising diagnostics, therapeutics, and research. The embodiments disclosed herein are aimed at fulfilling these and other needs in the art.

### SUMMARY

The present disclosure relates to novel topical exosome compositions and methods of making and using the same. Exemplary compositions disclosed herein include (1) a hair loss solution comprising minoxidil, hydroxypropyl-β-cyclodextrin (HPbCD), milk exosomes including peptide/protein fractions, PEG 6000 solution, and any combination thereof; (2) a skin lightening moisturizing cream for normal to dry skin comprising hydroquinone, Vitamins A (retinol) and B3, arbutin, kojic acid, sun protection factor (SPF) and any combination thereof; (3) an anti-wrinkle eye cream comprising milk exosomes, vanishing cream, ferulic acid, hyaluronic acid, retinol, and any combination thereof; (4) a rejuvenating eye cream comprising mesenchymal stem cells (MSC) exosome, secretome, and any combination thereof; (5) a facial skin rejuvenation cream comprising MSC and Milk exosomes, and any combination thereof; (6) an anti-aging composition (antioxidants, aka Serum) comprising retinols, milk exosomes and peptides (collagen stimulants), A-MSC exosomes under hyperbaric O₂, and any combination thereof; (7) a wound healing cream comprising platelet-rich plasma (PRP), induced pluripotent stem cells (iPCs), MSC exosomes, oil in water (O/W) cream, and any combination thereof; (8) a diabetic wound treatment comprising Hyperbaric CO₂ induced MSC exosomes (angiogenic factors; overexpressing Nrf2), and any combination thereof; (9) an atopic dermatitis treatment comprising A-MSC exosomes and any combination thereof; (10) a moisturizing day/night lip cream or balm comprising rejuvenating and antioxidant factors such as exosomes and any combination thereof; (11) a skin tanning moisturizing cream for normal to dry skin comprising dihydroxyacetone (DHA), Vitamins A (retinol) and/or B3 and any combination thereof; (12) a facial/eye mask comprising rejuvenating and antioxidant factors such as exosomes and any combination thereof; and (13) an Acne Rosacea treatment comprising exosomes loaded with Azelaic acid, Isotretinoin and/or Retinoic acid and any combination thereof. The exemplary embodiments disclosed herein in connection with particular uses are not necessarily limited to said use(s), and multiple uses for the disclosed compositions are envisioned and within the scope of the present disclosure.

### BRIEF DESCRIPTION OF THE DRAWINGS

For a fuller understanding of the embodiments disclosed herein, reference is made to the following detailed description, taken in connection with the accompanying drawings illustrating various embodiments of the present disclosure, in which:

FIG. 1A depicts western blot chemiluminescence curves for exosomes prepared in accordance with the teachings of the present disclosure;

FIG. 1B depicts a quantitative analysis of the chemiluminescence curves of FIG. 1A;

FIG. 1C depicts the western blots used to generate the data of FIGS. 1A and 1B;

FIG. 2A depicts western blot chemiluminescence curves for exosomes prepared in accordance with the teachings of the present disclosure;

FIG. 2B depicts a quantitative analysis of the chemiluminescence curves of FIG. 2A;

FIG. 2C depicts the western blots used to generate the data of FIGS. 2A and 2B;

FIG. 3A depicts western blot chemiluminescence curves for exosomes prepared in accordance with the teachings of the present disclosure;

FIG. 3B depicts a quantitative analysis of the chemiluminescence curves of FIG. 3A;

FIG. 3C depicts the western blots used to generate the data of FIGS. 3A and 3B;

FIG. 4A depicts western blot chemiluminescence curves for exosomes prepared in accordance with the teachings of the present disclosure;

FIG. 4B depicts a quantitative analysis of the chemiluminescence curves of FIG. 4A;

FIG. 4C depicts the western blots used to generate the data of FIGS. 4A and 4B;

FIG. 5A depicts the results of an MTT Assay after treatment of BJ fibroblasts with A-MSC exosomes and secretome prepared in accordance with the teachings of the present disclosure;

FIG. 5B depicts the results of an MTT Assay after treatment of HEK293 cells with A-MSC exosomes and secretome prepared in accordance with the teachings of the present disclosure;

FIG. 6A depicts the results of a WST Assay 24 hours after treatment of BJ fibroblasts with A-MSC exosomes and secretome prepared in accordance with the teachings of the present disclosure;

FIG. 6B depicts the results of a WST Assay 24 hours after treatment of HEK293 cells with A-MSC exosomes and secretome prepared in accordance with the teachings of the present disclosure;

FIG. 6C depicts the results of a WST Assay 48 hours after treatment of BJ fibroblasts with A-MSC exosomes and secretome prepared in accordance with the teachings of the present disclosure;

FIG. 6D depicts the results of a WST Assay 48 hours after treatment of HEK293 cells with A-MSC exosomes and secretome prepared in accordance with the teachings of the present disclosure; and

FIG. 7 depicts the results of a scratch assay on BJ fibroblasts treated with A-MSC exosomes and secretome.

### DETAILED DESCRIPTION OF EMBODIMENTS

The present disclosure relates to exosome-based topical compositions and methods for making and using the same, including using the compositions for the treatment of a variety of conditions. It is to be understood that the descriptions of the present embodiments have been simplified to illustrate elements that are relevant for a clear understanding, while eliminating, for the purposes of clarity, many other elements that may be found in the present embodiments. Those of ordinary skill in the pertinent art will recognize that other elements are desirable and/or required in order to implement the present embodiments. However, because such elements are well known in the art, and because such elements do not facilitate a better understanding of the present embodiments, a discussion of such elements is not provided herein. Exosomes employed in connection with embodiments of the present disclosure may be bovine milk derived, MSC exosomes, A-MSC exosomes, HEK293 exosomes, NSC exosomes or any other exosome type suitable for the delivery purposes described herein that may be loaded by different methods or used as their natural form. The exosomes may be prepared in accordance with existing techniques known in the art. Alternatively, the exosomes may be prepared in accordance with one or more of the methods disclosed herein. In one or more embodiments, exosomes may be loaded with drugs such as, for example, minoxidil, hydroquinone, corticosteroids, or biologicals or natural compounds drug-excipient complexes. Drug may mean any biological or chemical active pharmaceutical ingredient or natural product or new candidate. Drug delivery may target skin layers such as epidermis, dermis or hypodermis or intended and employed for systemic drug delivery. Dosage forms for any one of the embodiments disclosed herein may include, but are not limited to, ointment, cream, gel, topical spray, patch, emulsion, suspension, solution and/or powder. Other pharmaceutical dosage forms may also be used to deliver drugs including but not limited to injectables for dermal drug delivery.

Reference throughout this specification to “one embodiment”; “an embodiment”; “one or more embodiments”; or the like means that a particular feature, structure, step, or characteristic described in connection with the embodiment is included in at least one embodiment. Thus, appearances of the phrases “in one embodiment”; “in an embodiment”; “one or more embodiments”; or the like in various places throughout this specification are not necessarily all referring to the same embodiment. Moreover, the individual elements of a composition or steps in a method may be interchanged between the various exemplary embodiments without departing from the scope of the present disclosure.

In addition, for the purpose of promoting an understanding of the principles of the present disclosure, reference will now be made to the embodiments described herein, and specific language will be used to describe the same. It will, nevertheless, be understood that no limitation of the scope of the disclosure is thereby intended, and any alterations and further modifications of the described or illustrated embodiments and any further applications of the principles of the disclosure as illustrated herein are contemplated as would normally occur to one skilled in the art to which the disclosure relates. All limitations of scope should be determined in accordance with and as expressed in the eventual claims of one or more issued patents.

Unless defined otherwise, all technical and scientific terms used herein have the same meaning as commonly understood by one of ordinary skill in the art to which the subject matter of this disclosure belongs. Although any methods and materials similar or equivalent to those described herein can be used in the practice or testing of the present embodiments, exemplary preferred methods and materials are described.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, encompasses variations of ±20%, or ±10%, or ±5%, or ±1%, or ±0.1% from the specified value, as such variations are appropriate to prepare the disclosed compositions and perform the disclosed methods.

Throughout this disclosure, various aspects of the embodiments are presented in a range format. It should be understood that the description in range format is merely for convenience and brevity and should not be construed as an inflexible limitation on the scope of the embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 2, 1 to 3, from 1 to 4, from 1 to 5, from 2 to 4, from 2-5, from 2 to 6, from 3 to 6 and so on, as well as individual numbers within that range, for example, 1, 2, 2.7, 3, 4, 5, 5.3, and 6. This applies regardless of the breadth of the range.

The following techniques may be employed in connection with one or more embodiments of the present disclosure. One of ordinary skill in the art, having the benefit of the present disclosure, will appreciate that other known techniques may be employed without departing from the spirit and scope of the present disclosure.

Exemplary Method for Preparing Exosomes

While at least one exemplary protocol for the manufacture of exosomes is provided herein, one of ordinary skill in the art, having the benefit of the present disclosure, will appreciate that alternative methods for purifying and/or preparing exosomes may be employed without departing from the scope of the present disclosure. Accordingly, the compositions and methods disclosed herein are not necessarily limited to use of the disclosed methods for preparing exosomes.

By way of non-limiting example, A-MSC cultures may be scaled from 1 liter to 5 liters and then 30 liters. We evaluate both fed batch and multiple conditioning run methods of culture at 30 liters. The yield is optimized using the final exosomal yield (EVs/ml of conditioned medium) conforming to reference standard criteria. The conformance is checked against release criteria (Table 1). The criteria for success include the quantitative milestones listed below. EVs are purified from the 30 liters of conditioned media using filtration followed by multimodal chromatography. We typically produce at least 5E13 EVs per liter of conditioned media following nutrient supplement deprivation. However, other stressing regimens such as oxidative stress or chemicals are also employed.

The following quantitative milestones are assessed. (1) Culture 30 liters of A-MSC cells on microcarriers in bioreactor. The bioreactors may include stirred tank, rocking bed bioreactor, wave bioreactor, packed bed bioreactor, circulation bioreactor or vertical wheel bioreactor. EVs are purified using filtration with the following criteria: (2) Produce EVs with 90% having a size of 100 nm (±80-120 nm). (3) Generate yields of at least 5E13 EVs per L of conditioned media, (4) Contain protein not greater than 12.5 ug protein per ug of EVs, (5) Identify the presence of a lipid bilayer in the EVs using electron microscopy, (6) Detectable presence of a minimum of three of the following EV-specific proteins (CD9, CD63, CD81, TSG101 and Annexin A1) by Western blot (lacking Calreticulin and GAPDH) and (7) Batch to batch variation of not more that 10% based upon the technical feedback from the center for biologics research and evaluation at the FDA.

To purify the exosomes in the downstream processing, methods employed include ways to isolate and purify EVs using spiral column filtration, multimodal column chromatography, viral deactivation and polishing rather than the traditional ultracentrifugation process. Spiral column filtration using 500-750 KDa cutoffs is used to concentrate and diafiltrate the conditioned medium. The retentate is chromatographed against anion exchange and size exclusion columns or a bimodal column. Column packing and eluent properties are optimized to improve the yield. The filtrate is filtered through 3-30 KDa dead-end or hollow fiber filter to concentrate the secretome to process further to isolate the growth factors. Acidic pH adjustment (˜2-3.5) is used to deactivate the viruses. Further diafiltration against PBS is included to gain the final product concentration.

TABLE 1
Exemplary Quality Control and Release Criteria.

| | Release criteria | |
|---|---|---|
| Parameter | (range or profiles) | Method |
| Parental cells | | |
| Cell count and | ≥90% viable cells | Manual count, |
| viability | | Trypan blue method |
| Cell surface | ≥95% CD29⁺, CD44 [symbol] , | Multi-color flow |
| marker profile | CD73 [symbol] , CD99 [symbol] , | cytometry |
| | CD105 [symbol] , CD [symbol] | |
| | ≤2.5% CD14 [symbol] , CD19 [symbol] , | |
| | CD34 [symbol] , CD45 [symbol] , | |
| | MHC class II [symbol] | |
| Identity purity | | |
| and impurities | | |
| EV quantity | 4E11 EVs/ml | NTA |
| EV size | 80-120 nm | NTA |
| EV particle | Percentage of CD63+, | Fluorescent |
| identity | CD [symbol] 1+, CD73+ | channel NTA |
| | (≥10-15%) | |
| EV Surface | CD63 [symbol] , CD81 [symbol] , | Western blot |
| Marker Profile | Tsg101⁺ | |
| | CD [symbol] , CD29 [symbol] , | Flow cytometry |
| | CD44 [symbol] , CD 49e [symbol] , | MACS Plex |
| | CD63 [symbol] , CD81 [symbol] , | |
| | CD73 [symbol] , CD105 [symbol] , | |
| | MCSP [symbol] | |
| | CD14 [symbol] , CD19 [symbol] , | |
| | CD34 [symbol] , CD45 [symbol] , | |
| | CD142 [symbol] , MHC class I [symbol] , | |
| | class II [symbol] | |
| EV protein | μg/EV particle | Bradford-NTA |
| concentration | | |
| RNA | μg/EV particle | RNA assay kit |
| concentration | | |
| EV m [symbol] RNA/RNA | Profile conforms to reference | qRT-PCR |
| profile | standard | |
| Microbial | | |
| Impurities | | |
| EV endotoxin | <5 EU/kg bodyweight of the | Tests according |
| | recipient | to USP |
| EV sterility | Negative | Tests according |
| | | to USP |
| EV mycoplasma | Negative | Tests according |
| | | to USP |
| Reference | | |
| standard | | |
| specific | | |
| Structural integrity | Spherical and size | Cryo-TEM |
| | (80-120 nm) | |
| Proteomic profile | Contains defined profile | LC-MS/MS |
| Lipidomic profile | Contains defined profile | LC-MS/MS |
| Cytokine profile | Contains defined profile | LC-MS/MS, ELISA |
| | | or Multiplex |
| | | assay; RayBioR |
| | | Cytokine Antibody |
| | | Array or Luminex |
| | | XMaxR antibody |
| | | bead-based |
| | | assay |
| Bioassays | | |
| Enhanced cell | Conforms (Pvalue < 0.06) | In vitro/in vivo |
| proliferation | | potency assays |
| Inhibition of | Conforms (Pvalue < 0.06) | In vitro/in vivo |
| T-cell growth | | potency assays |
| Endothelial cell | Conforms (Pvalue < 0.0 [symbol] ) | In vitro/in vivo |
| growth assay | | potency assays |
| Neuroprotective | Conforms (Pvalue < 0.0 [symbol] ) | In vitro/in vivo |
| activity | | potency assays |
| Anti- [symbol] | Conforms (Pvalue < 0.0 [symbol] ) | In vitro/in vivo |
| activity | | potency assays |
| Anti-inflammatory | Conforms (Pvalue < 0.0 [symbol] ) | In vitro/in vivo |
| activity | | potency assays |
| Accelerated bone | Conforms (Pvalue < 0.06) | In vitro/in vivo |
| healing | | potency assays |

[symbol]

indicates data missing or illegible when filed

30 liters of culture media of A-MSCs containing at least 1E13 of EV/L is collected and subjected to in-depth filtration series 25 μm, 10 μm, 4 μm, 1 μm, 0.22 μm. The filtrate is concentrated to 600 ml using hollow fiber cartridge filtration (GE) with 500-750 KDa cut-off filters. In order to remove DNA, the concentrated sample (50×) is treated with Benzonase endonuclease. Benzonase endonuclease degrades all nucleic acids down to oligonucleotides of approximately 3 to 5 base pairs in length. These small fragments are removed by next step diafiltration and also can enter into the core of chromatography system where they are bound by the octylamine ligands. The Benzonase endonuclease itself can also be removed by chromatography system. Then, the sample is subjected to 10-times diafiltration using PBS. The diafiltrated sample is chromatographed against a multimodal chromatography resin. 300 ml of resin is packed in a column and equilibrated with PBS. The EV-rich sample (600 ml) is loaded onto the column at a flow rate of 20 ml/min. After running the sample, the column is washed with 300 ml of PBS buffer and then with 0.1M NaOH in 27% 1-propanol as cleaning in place (CIP) solution. The pH of CIP fractions are immediately adjusted to 7.5 with 0.1M HCl in order to analyze the bound proteins. Chromatography column is regenerated with 1M NaOH in 27% 1-propanol solution. 10 ml fractions are collected at each sample loading step to evaluate purified EV and also at elution step to analysis of bounded proteins. Particle size and concentration of EVs are determined via nanoparticle tracking analysis (NTA). Protein quantities in samples are measured using bicinchoninic acid assay kit and RNA concentration are quantified using the RNA assay kit. Western blots are performed in order to test for the presence of surface markers CD9, CD63, CD81, TSG101 and Annexin A1. Image analysis is performed using Image J software. Acidic pH adjustment (3.5) for 1 hour is used to deactivate the viruses. Further diafiltration against PBS is included to gain the final product concentration on a hollow fiber or spiral column filter system.

Exosomes may be lyophilized using cryoprotectants and stabilizers such as proteins (peptides), cyclodextrin, or sugars. Process parameters are temperature (deep freeze, −50 to −85° C.), vacuum (50-200 mTorr), and drying time (36-72 h) depending on the solution buffer and exosome concentration.

Second Exemplary Method for Preparing Exosomes

A-MSC cells are cultured in T-25, T-75 flasks, then transferred to 100 ml bioreactor on Cytodex 1 or 3 microcarriers. The cells are cultured for 7 days in 250 ml and 500 ml bioreactors and conditioned for 2 days. The conditioning regimen included replacing the media with the basal media and removing any supplements and growth factors. After media harvest, the conditioned medium was filtered through 20 um, 10 um, 4 um and 0.22 um filters. The cleared conditioned media was (ultra)-centrifuged at the following: 300 g for 10 min; 2000 g for 20 min; 10,000 g for 30 min; and 100,000 g for 4 h or 120,000 g for 2 h or 150,000 g for 1 h. Then the pellet is resuspended in PBS for storage at −80° C. Alternatively, 750 KDa filtration could be used to prepare the exosomes crude samples and then run the ultracentrifugation step or run Capto Core multimodal chromatography.

Exemplary Method of Secretome Preparation.

The method for exosome preparation was used with added steps for secretome preparation. The supernatant after ultracentrifugation was passed through 3 KDa filter and the retentate was collected. The concentration ratio ranged from 35 to 50 times the initial volume.

Exemplary Methods for Loading of Exosomes with Drugs or Other Components

The loading of exosomes with drugs can be achieved with various known methods. The choice of method is often dependent on the physical properties of the drug of interest. For instance, a drug with an overall negative charge may be very difficult to load into exosomes using a passive method because exosomes carry an overall negative charge.

Therefore, one or more embodiments of the present disclosure may utilize electroporation to load exosomes with various chemical components. By way of non-limiting example, an electroporation method may be used to load drug(s) or other components into exosomes, but in order to do so, exosomes need to be previously reconstituted and frozen away in Optiprep-free (see recipe below) electroporation buffer. Large numbers (e.g., 5×10⁹ to 5×10¹⁰) of exosomes are brought to 1× Optiprep using the 2× Optiprep buffer, and the total reaction mix is brought up to 400 ul with drug and 1× Optiprep Electroporation buffer. Note that, in one or mpore embodiments, the required drug volume should be as low as possible such that the buffer is not compromised; if drug volume is excessive, consider using 2× buffer to compensate.

Exemplary Electroporation Buffer Recipe:

Components

3M KCl (Mettler Toledo #63056166), 0.22 um filtered

0.5 M Potassium Phosphate, pH 7.2 (Alfa Aesar #J60326), 0.22 um filtered

OptiPrep (Stemcell Technologies #07820), sterile

Water (Hyclone Molecular Biology Grade), sterile

Electroporation Buffer

57.5 ul 0.5M K-Phosphate

208.3 ul 3M KCl

5.25 ml OptiPrep

19.45 ml Water

Note that this is 1× OptiPrep. When used to reconstitute EVs, one would have to do so with buffer without OptiPrep (OptiPrep should not be frozen); 2× OptiPrep would be required when using your frozen EVs such that the final OptiPrep concentration is 1× in your electroporation buffer. Exemplary electroporation reaction conditions (400 ul total volume) comprise 0.4 cm electroporation cuvettes, 400V, 125 uF.

An alternative exemplary method for loading exosomes comprises an incubation protocol. Incubation is a passive method of loading drug into exosomes. Simply, drug and exosomes are suspended together and incubated until loading is maximized. This works well for neutral or positively charged drugs. We have successfully loaded exosomes suspended in DPBS with drug at various temperatures (37° C. and 4° C., for example). Also, loading time may vary, and we have used loading times as low as 5 minutes, and loading times of 18 hours.

Yet another exemplary exosome loading protocol may utilize commercially available reagents such as Lipofectamine (ThermoFisher) and Exo-Fect (SBI), which are practical options to use in cases where exosome loading is challenging. Size and/or charge on drugs often makes exosome loading difficult, so using commercial reagents should be kept in mind in such cases.

Other exemplary loading methods are available and can be viable options. Methods such as sonication and extrusion are methods used with success. Also, combinations of methods may be utilized, such as, for example, using commercial reagents combined with electroporation.

### Exemplary Embodiment 1

One or more embodiments of the present disclosure provide a composition for treating hair loss comprising minoxidil; hydroxypropyl-β-cyclodextrin (HPbCD); milk exosomes including peptide/protein fractions; and MC, PG, PEG 6000 and/or any combination thereof. In one or more exemplary embodiments, the composition comprises about 5% minoxidil; about 2% hydroxypropyl-β-cyclodextrin (HPbCD; milk exosomes including peptide/protein fractions); (MC, PG, PEG 6000); and any combination thereof. Alternatively, one or more exemplary embodiments may comprise minoxidil powder USP; ethanol absolute; hydroxypropyl-β-cyclodextrin (HPβCD) pharmaceutical grade; propylene glycol (preservative; stabilizing agent; cosolvent); glycerin; DL-Lactic acid; bovine raw milk exosomes; bovine raw milk ultracentrifugation peptide/protein fraction and/or 750 KDa TFF filtrate (100 KDa retentate); MSC exosomes; MSC secretome (MSC culture conditioned medium; TFF 750 KDa filtrate-3 KDa retentate); methyl cellulose (MC); and/or Polyethylene Glycol 6000 and any combination thereof.

By way of non-limiting example, one or more exemplary embodiments of the present disclosure comprise 5 g of minoxidil powder USP; 1-5 ml of Ethanol absolute; 20-35 g of hydroxypropyl-β-cyclodextrin (HPβCD) pharmaceutical grade; 5-10 ml of propylene glycol (Preservative; stabilizing agent; cosolvent); 5-20 ml of glycerin; 2.5-5 ml of DL-Lactic acid; 1 ml of bovine raw milk exosomes (1E12 EVs/ml); 100 ml of bovine raw milk ultracentrifugation peptide/protein fraction or 750 KDa TFF filtrate (100 KDa retentate); MSC exosomes (1E12 EVs/ml); MSC secretome (MSC culture conditioned medium; TFF 750 KDa filtrate-3 KDa retentate); methyl cellulose (MC) (1-2%); polyethylene glycol 6000 (up to 10% W/V) to achieve viscosity of 15-25 cP; and any combination thereof.

Exemplary methods for preparing the disclosed hair loss treatment compositions are herein provided. One of ordinary skill in the art, having the benefit of the present disclosure, will appreciate that alternative methods may be available without departing from the spirit and scope of the present disclosure.

### Exemplary Method 1.1

One or more embodiments of the present disclosure comprise at least one of (i) dissolving 20-35 g of hydroxypropyl-β-cyclodextrin (HPβCD) in 75 ml of WFI water and mixing at 70 rpm for 5 min at room temperature; (ii) dispersing 5 g of minoxidil powder in 5 ml ethanol (70%) on stirring at 100 rpm, wherein when the minoxidil is dissolved; (iii) adding the solution to 10 ml of bovine raw milk ultracentrifugation peptide/protein fraction or 750 KDa TFF filtrate (100 KDa retentate) on stirring at 100 rpm at room temperature; (iv) mixing the solution for 15 min at 100 rpm; (v) cooling the sample to about 8° C.; (vi) adding 1 ml of bovine raw milk exosomes (1E12 EVs/ml) on stirring at 45 rpm at about 4-8° C.; (vii) adding bovine raw milk ultracentrifugation peptide/protein fraction or 750 KDa TFF filtrate (100 KDa retentate) up to a total volume of 100 ml; and (viii) adding methyl cellulose (MC) (1-2%) or polyethylene glycol 6000 (up to 10% W/V) to achieve a viscosity of about 15-25 cP.

### Exemplary Method 1.2

One or more embodiments of the present disclosure comprise at least one of (i) dissolving 20-35 g of hydroxypropyl-β-cyclodextrin (HPβCD) in 75 ml of bovine raw milk ultracentrifugation peptide/protein fraction or 750 KDa TFF filtrate (100 KDa retentate) and mixing at 70 rpm for 5 min at room temperature (mixture I); (ii) dissolving 5 g of minoxidil powder in 5 ml ethanol (70%) on stirring at 100 rpm at room temperature; (iii) adding 5 ml of propylene glycol and continuing mixing, wherein when minoxidil is dissolved; (iv) adding the solution to mixture I on stirring at 100 rpm at room temperature; (v) mixing the solution for 15 min at 100 rpm; (vi) adding 1 ml of bovine raw milk exosomes (1E12 EVs/ml) on stirring at 45 rpm at 16° C.; (vii) adding bovine raw milk ultracentrifugation peptide/protein fraction or 750 KDa TFF filtrate (100 KDa retentate) up to a total volume of 100 ml; and (viii) adding methyl cellulose (MC) (1-2%) or polyethylene glycol 6000 (up to 10% W/V) to achieve viscosity of 15-25 cP.

### Exemplary Method 1.3

One or more embodiments of the present disclosure comprise at least one of (i) dissolving 20-35 g of hydroxypropyl-β-cyclodextrin (HPβCD) in 75 ml of bovine raw milk ultracentrifugation peptide/protein fraction or 750 KDa TFF filtrate (100 KDa retentate) and mixing at 70 rpm for 5 min at room temperature (mixture I); (ii) dissolving 5 g of Minoxidil powder in 5 ml ethanol (70%) on stirring at 100 rpm at room temperature; (iii) adding 5 ml of glycerin and continuing mixing (mixture II) (iv) mixing mixtures I and II for 15 min at 50 rpm; (v) adding 1 ml of bovine raw milk exosomes (1E12 EVs/ml) on stirring at 45 rpm in 16° C.; (vi) adding bovine raw milk ultracentrifugation peptide/protein fraction or 750 KDa TFF filtrate (100 KDa retentate) up to a total volume of 100 ml; and (vii) adding methyl cellulose (MC) (1-2%) or polyethylene glycol 6000 (up to 10% W/V) to achieve viscosity of 15-25 cP.

### Exemplary Method 1.4

One or more embodiments of the present disclosure comprise at least one of (i) dissolving 20-35 g of hydroxypropyl-β-cyclodextrin (HPβCD) in 75 ml of bovine raw milk ultracentrifugation peptide/protein fraction or 750 KDa TFF filtrate (100 KDa retentate) and mixing at 70 rpm for 5 min at room temperature (mixture I); (ii) dissolving 5 g of minoxidil powder in 5 ml ethanol (100%) on stirring at 100 rpm at room temperature; (iii) adding 5 ml of glycerin and continuing mixing; (iv) adding 2.5 ml of Lactic acid and continuing mixing (mixture II); (v) mixing solutions I and II for 15 min at 50 rpm; (vi) adding 1 ml of bovine raw milk exosomes (1E12 EVs/ml) on stirring at 45 rpm in 16° C.; adding 1 ml of MSC exosomes (1E12 EVs/ml) on stirring at 45 rpm in 16° C.; (vii) adding bovine raw milk ultracentrifugation peptide/protein fraction or 750 KDa TFF filtrate (100 KDa retentate) up to a total volume of 100 ml; and (viii) adding methyl cellulose (MC) (1-2%) or polyethylene glycol 6000 (up to 10% W/V) to achieve a viscosity of 15-25 cP.

### Exemplary Method 1.5 (Alcohol and Propylene Glycol Free Formulation)

One or more embodiments of the present disclosure comprise at least one of (i) dissolving 20-35 g of hydroxypropyl-β-cyclodextrin (HPβCD) in 75 ml of bovine raw milk ultracentrifugation peptide/protein fraction or 750 KDa TFF filtrate (100 KDa retentate) and mixing at 70 rpm for 5 min at room temperature (mixture I); (ii) dissolving 5 g of minoxidil powder in mixture I on stirring at 100 rpm at room temperature for 6 h; (iii) adding 5 ml of glycerin and continuing mixing; (iv) adding 1 ml of bovine raw milk exosomes (1E12 EVs/ml) on stirring at 45 rpm in 16° C.; (v) adding 1 ml of MSC exosomes (1E12 EVs/ml) on stirring at 45 rpm in 16° C.; (vi) adding 15 ml of MSC secretome (750 KDa filtrate-3 KDa retentate) on stirring at 45 rpm at 16° C.; (vii) adding bovine raw milk ultracentrifugation peptide/protein fraction or 750 KDa TFF filtrate (100 KDa retentate) up to a total volume of 100 ml; and (viii) adding methyl cellulose (MC) (1-2%) or polyethylene glycol 6000 (up to 10% W/V) to achieve viscosity of 15-25 cP.

### Exemplary Embodiment 2

One or more embodiments of the present disclosure provide a composition and/or cream for skin lightening (brightening or whitening) comprising hydroquinone; vitamin A (retinol); vitamin B3; arbutin; kojic acid; and/or viola tricolor ethanolic extract and any combination thereof. In one or more exemplary embodiments, the composition comprises about 4% hydroquinone; vitamin A (retinol); vitamin B3; arbutin; kojic acid; and/or sun protection factor (SPF) and any combination thereof. Alternatively, one or more embodiments of the present disclosure may comprise O/W base cream (such as, for example, Humco PENcream™, Humco MultiBase™ Humco SaltStable LO™); Hydroquinone; Vitamin A (as retinol); Vitamin B3; Arbutin; Kojic acid as Kojic dipalmitate; bovine raw milk exosomes (1E12 EVs/ml); bovine raw milk ultracentrifugation peptide/protein fraction or 750 KDa TFF filtrate (100 KDa retentate); MSC exosomes; MSC secretome (MSC culture conditioned medium; TFF 750 KDa filtrate-3 KDa retentate); and any combination thereof.

By way of non-limiting example, one or more exemplary embodiments of the present disclosure comprise O/W base cream (such as, for example, Humco PENcream™, Humco MultiBase™, Humco SaltStable LO™); 2.4 g of Hydroquinone; Vitamin A (as retinol) 2.5 g/100 g; Vitamin B3 5 g/100 g; Arbutin as 5 g/100 g; Kojic acid as Kojic dipalmitate 0.2 g/100 g; 0.6 ml of bovine raw milk exosomes (1E12 EVs/ml); 5 ml of bovine raw milk ultracentrifugation peptide/protein fraction or 750 KDa TFF filtrate (100 KDa retentate) 50× concentrate; 0.6 ml of MSC exosomes (1E12 EVs/ml); and/or 1 ml of MSC secretome (MSC culture conditioned medium; TFF 750 KDa filtrate-3 KDa retentate) 50× concentrate and any combination thereof.

Exemplary methods for preparing the disclosed skin-lightening compositions are herein provided. One of ordinary skill in the art, having the benefit of the present disclosure, will appreciate that alternative methods may be available without departing from the spirit and scope of the present disclosure.

### Exemplary Method 2.1

One or more embodiments of the present disclosure comprise at least one of (i) selecting an O/W base cream from a commercial source; (ii) providing 30 g of the base cream at ambient temperature; (iii) dissolving 2.4 g of hydroquinone in 3 ml of alcohol; (iv) adding the solution to 30 g of base cream and mixing at 50 rpm in 45° C. for 15 min; (v) adding Vitamin A (as retinol) 2.5 g/100 g, Vitamin B3 5 g/100 g, Arbutin 5 g/100 g, and Kojic acid as Kojic dipalmitate 0.2 g/100 g and mixing at 50 rpm at 45° C. for 10 min; (vi) adding 0.6 ml of bovine raw milk exosomes (1E12 EVs/ml) and 5 ml of bovine raw milk ultracentrifugation peptide/protein fraction or 750 KDa TFF filtrate (100 KDa retentate) 50× concentrate; (vii) mixing at 45 rpm at 20° C. for 10 min; and (viii) adjusting to 60 g using base cream.

### Exemplary Method 2.2

One or more embodiments of the present disclosure comprise at least one of (i) selecting an O/W base cream from commercial source; (ii) providing 30 g of the base cream at ambient temperature; (iii) dissolving 2.4 g of hydroquinone in 3 ml of alcohol; (iv) adding the solution to 30 g of base cream and mixing at 50 rpm at 45° C. for 15 min; (v) adding Vitamin A (as retinol) 2.5 g/100 g, Vitamin B3 5 g/100 g, Arbutin 5 g/100 g, and Kojic acid as Kojic dipalmitate 0.2 g/100 g and mixing at 50 rpm in 45° C. for 10 min; (vi) adding 0.6 ml of bovine raw milk exosomes (1E12 EVs/ml), 2.5 ml of bovine raw milk ultracentrifugation peptide/protein fraction or 750 KDa TFF filtrate (100 KDa retentate) 50× concentrate, 0.6 ml of MSC exosomes (1E12 EVs/ml), and 1 ml of MSC secretome (MSC culture conditioned medium; TFF 750 KDa filtrate-3 KDa retentate) 50× concentrate and mixing at 45 rpm at 20° C. for 10 min; and (vii) adjusting to 60 g using base cream.

### Exemplary Method 2.3

One or more embodiments of the present disclosure comprise at least one of (i) selecting an O/W base cream from commercial source; (ii) providing 30 g of the base cream at ambient temperature; (iii) dissolving Viola tricolor ethanolic extract in appropriate solvent and adding to the base; (iv) adding the solution to 30 g of base cream and mixing at 50 rpm at 45° C. for 15 min; (v) adding Vitamin A (as retinol) 2.5 g/100 g and Vitamin B3 5 g/100 g and mixing at 50 rpm at 45° C. for 10 min; (vi) adding 0.6 ml of bovine raw milk exosomes (1E12 EVs/ml), 5 ml of bovine raw milk ultracentrifugation peptide/protein fraction or 750 KDa TFF filtrate (100 KDa retentate) 50× concentrate, 0.6 ml of MSC exosomes (1E12 EVs/ml), and 1 ml of MSC secretome (MSC culture conditioned medium; TFF 750 KDa filtrate-3 KDa retentate) 50× concentrate and mixing at 45 rpm at 20° C. for 10 min; and (vii) adjusting to 60 g using base cream.

### Exemplary Embodiment 3

One or more embodiments of the present disclosure provide a composition and/or cream for eye rejuvenation comprising MSC exosomes and secretome. In one or more exemplary embodiments, a composition comprises a vanishing base cream (supplied from commercial source; such as Humco PENcream™ or Humco MultiBase™); Vitamin A (as retinol); Vitamin B3; Vitamin E; Hydrolyzed collagen+hyaluronic acid (Cromoist HTA); Jojoba oil; Allantoin; Fibronectin; bovine raw milk exosomes; bovine raw milk ultracentrifugation peptide/protein fraction or 750 KDa TFF filtrate (100 KDa retentate); MSC exosomes; and MSC secretome (MSC culture conditioned medium; TFF 750 KDa filtrate-3 KDa retentate) and any combination thereof. In one or more embodiments, the MSC exosomes comprise A-MSC exosomes.

In one or more exemplary embodiments, a composition comprises a vanishing base cream (supplied from commercial source; such as Humco PENcream™ or Humco MultiBase™); Vitamin A (as retinol) 2.5 g/100 g; Vitamin B3 5 g/100 g; Vitamin E 50,000 IU/100 g; Hydrolyzed collagen+hyaluronic acid (Cromoist HTA) 2 g/100 g; Jojoba oil 2 g/100 g; Allantoin 0.5 g/100 g; Fibronectin 0.5 g/100 g; 0.5 ml of bovine raw milk exosomes (1E12 EVs/ml); 0.5 ml of bovine raw milk ultracentrifugation peptide/protein fraction or 750 KDa TFF filtrate (100 KDa retentate) 50× concentrate; 1 ml of MSC exosomes (1E12 EVs/ml); and 1 ml of MSC secretome (MSC culture conditioned medium; TFF 750 KDa filtrate-3 KDa retentate) 50× concentrate and any combination thereof.

Exemplary methods for preparing the disclosed eye-rejuvenating compositions are herein provided. One of ordinary skill in the art, having the benefit of the present disclosure, will appreciate that alternative methods may be available without departing from the spirit and scope of the present disclosure.

### Exemplary Method 3.1

One or more embodiments of the present disclosure comprise at least one of (i) selecting a vanishing base cream from commercial source such as Humco PENcream™ or Humco MultiBase™; (ii) providing 5 g of the base cream at ambient temperature; (iii) adding Vitamin A (as retinol) 0.25 g/10 g, Vitamin B3 0.5 g/10 g, E 5,000 IU/10 g, Hydrolyzed collagen+hyaluronic acid (Cromoist HTA) 0.2 g/10 g and mixing at 50 rpm at 45° C. for 10 min; (iv) adding 0.5 ml of bovine raw milk exosomes (1E12 EVs/ml), 0.5 ml of bovine raw milk ultracentrifugation peptide/protein fraction or 750 KDa TFF filtrate (100 KDa retentate) 50× concentrate, 1 ml of MSC exosomes (1E12 EVs/ml), and 1 ml of MSC secretome (MSC culture conditioned medium; TFF 750 KDa filtrate-3 KDa retentate) 50× concentrate and mixing at 45 rpm at 20° C. for 10 min; and (v) adjusting to 10 g using base cream.

### Exemplary Method 3.2

One or more embodiments of the present disclosure comprise at least one of (i) selecting a vanishing base cream from commercial source such as Humco PENcream™ or Humco MultiBase™; (ii) providing 5 g of the base cream at ambient temperature; (iii) adding Vitamin A (as retinol) 0.25 g/10 g, Vitamin B3 0.5 g/10 g, E 5,000 IU/10 g, Hydrolyzed collagen+hyaluronic acid (Cromoist HTA) 0.2 g/10 g and mixing at 50 rpm at 45° C. for 10 min; (iv) adding Jojoba oil 0.2 g/10 g, Allantoin 0.05 g/10 g, and Fibronectin 0.05 g/10 g; (v) adding (adjust concentrations and amounts as per need to make the formulation work) 0.5 ml of bovine raw milk exosomes (1E12 EVs/ml), 0.5 ml of bovine raw milk ultracentrifugation peptide/protein fraction or 750 KDa TFF filtrate (100 KDa retentate) 50× concentrate, 1 ml of MSC exosomes (1E11 EVs/ml), 1 ml of MSC secretome (MSC culture conditioned medium; TFF 750 KDa filtrate-3 KDa retentate) 50× concentrate and mixing at 45 rpm at 20° C. for 10 min; and (vi) adjusting to 10 g using base cream.

### Exemplary Method 3.3

One or more embodiments of the present disclosure comprise at least one of (i) selecting a vanishing base cream from commercial source such as Humco PENcream™ or Humco MultiBase™; (ii) providing 5 g of the base cream at ambient temperature; (iii) adding Vitamin A (as retinol) 0.25 g/10 g, Vitamin B3 0.5 g/10 g, E 5,000 IU/10 g, Hydrolyzed collagen+hyaluronic acid (Cromoist HTA) 0.2 g/10 g and mixing at 50 rpm in 45° C. for 10 min; (iv) adding Jojoba oil 0.2 g/10 g, Allantoin 0.05 g/10 g, and Fibronectin 0.05 g/10 g; (v) adding 1 ml of MSC exosomes (1E12 EVs/ml) and 1 ml of MSC secretome (MSC culture conditioned medium; TFF 750 KDa filtrate-3 KDa retentate) 50× concentrate and mixing at 45 rpm at 20° C. for 10 min; and (vi) adjusting to 10 g using base cream.

### Exemplary Embodiment 4

One or more embodiments of the present disclosure provide a composition for at least one of an anti-wrinkle and brightening eye serum comprising milk exosomes; vitamin C; ferulic acid; hyaluronic acid; Retinol; and any combination thereof. One or more embodiments of a composition may also comprise a vanishing base cream (supplied from commercial source; such as Humco PENcream™ or Humco MultiBase™); Vitamin A (as retinol); Vitamin B3; Vitamin E; Vitamin C (as Tetrahexyldecyl Ascorbate (THD Ascorbate); Magnesium ascorbyl phosphate (MAP); Ascorbyl 6 palmitate; Disodium isostearyl 2-0 L-ascorbyl phosphate (VCP-IS-Na); Tetraisopalmitoyl ascorbic acid (TIPA); Ascorbic acid sulfate; Coenzyme Q10 (Ubiquinone); Lactic acid; Ferulic Acid; Hydrolyzed collagen+hyaluronic acid (Cromoist HTA); Bovine raw milk exosomes; Bovine raw milk ultracentrifugation peptide/protein fraction or 750 KDa TFF filtrate (100 KDa retentate); MSC exosomes; MSC secretome (MSC culture conditioned medium; TFF 750 KDa filtrate-3 KDa retentate); and any combination thereof.

By way of non-limiting example, one or more exemplary embodiments of a composition comprise a vanishing base cream (supplied from commercial source; such as Humco PENcream™ or Humco MultiBase™); Vitamin A (as retinol) 2.5 g/100 g; Vitamin B3 5 g/100 g; Vitamin E 50,000 IU/100 g; Vitamin C (as Tetrahexyldecyl Ascorbate (THD Ascorbate); Magnesium ascorbyl phosphate (MAP); Ascorbyl 6 palmitate; Disodium isostearyl 2-0 L-ascorbyl phosphate (VCP-IS-Na); Tetraisopalmitoyl ascorbic acid (TIPA); Ascorbic acid sulfate) 15-20 g/100 g; Coenzyme Q10 (Ubiquinone) 3-6%; Lactic acid 1-5%; Ferulic Acid 0.8-1%; Hydrolyzed collagen+hyaluronic acid (Cromoist HTA) 2 g/100 g; Bovine raw milk exosomes (1E13 EVs/ml); Bovine raw milk ultracentrifugation peptide/protein fraction or 750 KDa TFF filtrate (100 KDa retentate) 50× concentrate; MSC exosomes (1E12 EVs/ml); MSC secretome (MSC culture conditioned medium; TFF 750 KDa filtrate-3 KDa retentate) 50× concentrate; and any combination thereof.

Exemplary methods for preparing the disclosed anti-wrinkle and brightening eye compositions are herein provided. One of ordinary skill in the art, having the benefit of the present disclosure, will appreciate that alternative methods may be available without departing from the spirit and scope of the present disclosure.

### Exemplary Method 4.1

One or more embodiments of the present disclosure comprise at least one of (i) selecting a vanishing base cream from a commercial source such as Humco PENcream™ or Humco MultiBase™ (ii) providing 5 g of the base cream at ambient temperature; (iii) adding Vitamin A (as retinol) 0.625 g/25 g, Vitamin B3 1.25 g/25 g, E 12,500 IU/25 g, Hydrolyzed collagen+hyaluronic acid (Cromoist HTA) 0.5 g/25 g and mix at 50 rpm in 45° C. for 10 min; (iv) adding the following: (adjust concentrations and amounts as per need) Vitamin C (as Tetrahexyldecyl Ascorbate (THD Ascorbate); Magnesium ascorbyl phosphate (MAP); Ascorbyl 6 palmitate; Disodium isostearyl 2-0 L-ascorbyl phosphate (VCP-IS-Na); Tetraisopalmitoyl ascorbic acid (TIPA); Ascorbic acid sulfate) 3.75-5 g/25 g; (v) adding Coenzyme Q10 (Ubiquinone) 0.75-1.5 g/25 g; (vi) adding a mixture of Lactic acid 1-5% and Ferulic acid 0.2-0.25 g/25 g; (vii) adding 0.5 ml of bovine raw milk exosomes (1E13 EVs/ml); (viii) adding 0.5 ml of bovine raw milk ultracentrifugation peptide/protein fraction or 750 KDa TFF filtrate (100 KDa retentate) 50× concentrate; (ix) adding 1 ml of MSC exosomes (1E12 EVs/ml); (x) adding 1 ml of MSC secretome (MSC culture conditioned medium; TFF 750 KDa filtrate-3 KDa retentate) 50× concentrate; (xi) mixing at 35 rpm in 16° C. for 10 min; and (xii) adjusting to 10 g using base cream.

### Exemplary Embodiment 5

One or more embodiments of the present disclosure provide a composition for facial skin rejuvenation comprising at least one of umbilical cord or other mesenchymal stem cells (MSC) and milk exosome. One or more embodiments of a composition may comprise a vanishing base cream (supplied from commercial source; such as Humco PENcream™ or Humco MultiBase™); Vitamin A (as retinol); Vitamin B3; Vitamin E; Bovine raw milk exosomes; Bovine raw milk Ultracentrifugation peptide/protein fraction or 750 KDa TFF filtrate (100 KDa retentate); Umbilical Cord MSC exosomes; Umbilical Cord MSC secretome (MSC culture conditioned medium; TFF 750 KDa filtrate-3 KDa retentate); and any combination thereof.

By way of non-limiting example, one or more embodiments of a composition may comprise a vanishing base cream (supplied from commercial source; such as Humco PENcream™ or Humco MultiBase™); Vitamin A (as retinol) 2.5 g/100 g; Vitamin B3 5 g/100 g; Vitamin E 50,000 IU/100 g; Bovine raw milk exosomes (5E13 EVs/ml); Bovine raw milk Ultracentrifugation peptide/protein fraction or 750 KDa TFF filtrate (100 KDa retentate) 50× concentrate; Umbilical Cord MSC exosomes (5E12 EVs/ml); Umbilical Cord MSC secretome (MSC culture conditioned medium; TFF 750 KDa filtrate-3 KDa retentate) 50× concentrate; and any combination thereof.

Exemplary methods for preparing the disclosed facial skin rejuvenation compositions are herein provided. One of ordinary skill in the art, having the benefit of the present disclosure, will appreciate that alternative methods may be available without departing from the spirit and scope of the present disclosure.

### Exemplary Method 5.1

One or more embodiments of the present disclosure may comprise at least one of (i) performing the steps according to at least one of exemplary methods 3.1, 3.2, and 3.3 described hereinabove; and (ii) adding 1 ml of bovine raw milk exosomes (5E13 EVs/ml); 0.5 ml of bovine raw milk ultracentrifugation peptide/protein fraction or 750 KDa TFF filtrate (100 KDa retentate) 50× concentrate; 1 ml of Umbilical Cord MSC exosomes (5E12 EVs/ml); 1 ml of Umbilical Cord MSC secretome (MSC culture conditioned medium; TFF 750 KDa filtrate-3 KDa retentate) 50× concentrate, and any combination thereof; (iii) mixing at 35 rpm at 16° C. for 10 min; and (iv) adjusting to 20 g using base cream.

### Exemplary Composition 6

Exosomes released by mouse mast cells exposed to oxidative stress differ in their mRNA content. Exosomes can influence the response of other cells to oxidative stress by providing recipient cells with a resistance against oxidative stress, observed as an attenuated loss of cell viability. Exposure to UV-light affects the biological functions associated with exosomes released under oxidative stress which happens also to the skin cells. Exosomes from bone marrow-derived mesenchymal stem cells (BMSCs) facilitate cell proliferation and survival by transporting various bioactive molecules, including microRNAs (miRs). BMSC-derived exosomes significantly decrease apoptosis rates and reactive oxygen species (ROS) production. A stronger effect is induced by exosomes collected from BMSCs cultured under hypoxic conditions than those collected from BMSCs cultured under normal conditions. Higher miR-214 enrichment in Hypoxic exosomes than in Normoxic exosomes. Mesenchymal stem cell-derived exosomes increase ATP levels, decrease oxidative stress and activate PI3K/Akt pathway.

However, the antioxidants released from astrocytes either via extracellular fluid or exosomes to neurons during stress conditions may not be sufficient to provide neuroprotection. Therefore, to combat oxidative stress in the central nervous system, synthetically developed exosomes loaded with antioxidants such as glutathione and the anti-aging protein Klotho may be employed.

Oxidative stress induction was simulated using a bioreactor to change the dissolved oxygen (DO %) in the medium. Normal dissolved oxygen (DO %), which ranges from about 30-50% in normoxic culture, was adjusted at 80-100% in oxidative stress induction conditions for varying time periods to evaluate the expression profile in the resulting exosomes, while all other process parameters were maintained at their optimum values. Downstream processes remained the same and the final exosomal and secretome were incorporated into the cosmeceutical preparation.

Accordingly, one or more embodiments of the present disclosure provide a composition for anti-aging (antioxidants, aka Serum) comprising retinols; milk exosomes and peptides (collagen stimulants); A-MSC exosomes under hyperbaric O₂; and any combination thereof.

By way of non-limiting example, one or more embodiments of the present disclosure may comprise a vanishing base cream (supplied from commercial source; such as Humco PENcream™ or Humco MultiBase™); Vitamin A (as retinol); Vitamin E; Bovine raw milk exosomes; Bovine raw milk ultracentrifugation peptide/protein fraction or 750 KDa TFF filtrate (100 KDa retentate); A-MSC exosomes at hyperbaric O₂ conditions; A-MSC secretome (MSC culture conditioned medium; TFF 750 KDa filtrate-3 KDa retentate) at hyperbaric O₂ conditions; and any combination thereof.

One or more embodiments of the present disclosure comprise a vanishing base cream (supplied from commercial source; such as Humco PENcream™ or Humco MultiBase™); Vitamin A (as retinol) 2.5 g/100 g; Vitamin E 50,000 IU/100 g; Bovine raw milk exosomes (5E13 EVs/ml); Bovine raw milk ultracentrifugation peptide/protein fraction or 750 KDa TFF filtrate (100 KDa retentate) 50× concentrate; A-MSC exosomes (5E12 EVs/ml) at hyperbaric O₂ conditions; A-MSC secretome (MSC culture conditioned medium; TFF 750 KDa filtrate-3 KDa retentate) 50× concentrate at hyperbaric O₂ conditions; and any combination thereof.

Exemplary methods for preparing the disclosed anti-aging compositions are herein provided. One of ordinary skill in the art, having the benefit of the present disclosure, will appreciate that alternative methods may be available without departing from the spirit and scope of the present disclosure.

### Exemplary Method 6.1

One or more embodiments of the present disclosure may comprise at least one of (i) performing the steps according to at least one of exemplary methods 3.1, 3.2, and 3.3 described hereinabove; (ii) adding 1 ml of bovine raw milk exosomes (5E13 EVs/ml), 0.5 ml of bovine raw milk ultracentrifugation peptide/protein fraction or 750 KDa TFF filtrate (100 KDa retentate) 50× concentrate, 1 ml of A-MSC exosomes (5E12 EVs/ml) at hyperbaric O₂ conditions, 1 ml of A-MSC secretome (MSC culture conditioned medium; TFF 750 KDa filtrate-3 KDa retentate) 50× concentrate at hyperbaric O₂ conditions; (iii) mixing at 35 rpm at 16° C. for 10 min; and adjusting to 20 g using base cream.

### Exemplary Embodiment 7

One or more embodiments of the present disclosure provide a composition for wound healing cream comprising platelet-rich plasma (PRP); induced pluripotent stem cells (iPCs); MSC exosomes; O/W cream; and any combination thereof.

One or more embodiments of the present disclosure comprise a vanishing base cream (supplied from commercial source; such as Humco PENcream™ or Humco MultiBase™); Vitamin A (as retinol); Vitamin E; PRP isolated exosomes; iPCs isolated exosomes; A-MSC exosomes at hyperbaric O₂ conditions; A-MSC secretome (MSC culture conditioned medium; TFF 750 KDa filtrate-3 KDa retentate) at hyperbaric O₂ conditions; and any combination thereof.

One or more embodiments of the present disclosure comprise a vanishing base cream (supplied from commercial source; such as Humco PENcream™ or Humco MultiBase™); Vitamin A (as retinol) 2.5 g/100 g; Vitamin E 50,000 IU/100 g; PRP isolated exosomes; iPCs isolated exosomes; A-MSC exosomes (5E12 EVs/ml) at hyperbaric O₂ conditions; A-MSC secretome (MSC culture conditioned medium; TFF 750 KDa filtrate-3 KDa retentate) 50× concentrate at hyperbaric O₂ conditions; and any combination thereof.

Exemplary methods for preparing the disclosed wound healing compositions are herein provided. One of ordinary skill in the art, having the benefit of the present disclosure, will appreciate that alternative methods may be available without departing from the spirit and scope of the present disclosure.

### Exemplary Method 7.1

One or more embodiments of the present disclosure may comprise at least one of (i) performing the steps according to at least one of exemplary methods 3.1, 3.2, and 3.3 described hereinabove; (ii) adding 1 ml of bovine raw milk exosomes (5E13 EVs/ml), 1 ml of A-MSC secretome (MSC culture conditioned medium; TFF 750 KDa filtrate-3 KDa retentate) 50× concentrate at hyperbaric O₂ conditions, PRP isolated exosomes, iPCs isolated exosomes; (iii) and mixing at 35 rpm at 16° C. for 10 min; and (iv) adjusting to 20 g using base cream.

### Exemplary Embodiment 8

Hyperbaric CO₂ induced MSC exosomes (angiogenic factors) MSC exosomes produced in hypoxia situation show different miRNA and protein profiles. The angiogenesis related factors are over-expressed in which different signaling pathways are involved including nuclear factor-kappaB signaling. Several putative paracrine effectors of angiogenesis present in MSC exosomes and increased in expression in MSCs exposed to hypoxia that include platelet derived growth factor, epidermal growth factor, overexpressing Nrf2, fibroblast growth factor, and most notably nuclear factor-kappaB (NFkB) signaling pathway proteins.

Overexpression of angiogenesis-related factors may be useful in stimulating vascularization in diabetic patients suffering from diabetic foot ulcer, which is caused by the effects of chronic ischemia, typically due to peripheral artery disease. These angiogenesis-related factors may stimulate pericytes to vascularize the affected tissue.

The hyperbaric CO₂ and/or hypoxia conditions were simulated using the bioreactor to change the CO₂% and/or dissolved oxygen (DO %) in the medium. Normal CO₂% range is typically about 5-10% during the experiments and/or dissolved oxygen (DO %) which ranged between about 30-50% in normoxic culture was adjusted to about 1-20% in hypoxic conditions. For the hyperbaric CO₂ induction, a range of about 10-40% was applied for varying time periods to evaluate the expression profile in the resulting exosomes, while all other process parameters were maintained at their optimum values. Downstream processes remained the same and the final exosomal and secretome was incorporated into the cosmeceutical preparation.

Accordingly, one or more embodiments of the present disclosure provide a composition for diabetic wound treatment comprising hyperbaric CO₂ induced MSC exosomes having at least one angiogenic factor and/or Nrf2 and any combination thereof.

One or more non-limiting embodiments may comprise a vanishing base cream (supplied from commercial source; such as Humco PENcream™ or Humco MultiBase™); Vitamin A (as retinol); Vitamin E; A-MSC exosomes at hyperbaric CO₂ or hypoxia conditions; A-MSC secretome (MSC culture conditioned medium; TFF 750 KDa filtrate-3 KDa retentate) at hyperbaric O₂ conditions at hyperbaric CO₂ or hypoxia conditions; and any combination thereof.

One or more non-limiting embodiments may comprise a vanishing base cream (supplied from commercial source; such as Humco PENcream™ or Humco MultiBase™); Vitamin A (as retinol) 2.5 g/100 g; Vitamin E 50,000 IU/100 g; A-MSC exosomes (5E12 EVs/ml) at hyperbaric CO₂ or hypoxia conditions; A-MSC secretome (MSC culture conditioned medium; TFF 750 KDa filtrate-3 KDa retentate) 50× concentrate at hyperbaric O₂ conditions at hyperbaric CO₂ or hypoxia conditions; and any combination thereof.

Exemplary methods for preparing the disclosed diabetic wound treatment compositions are herein provided. One of ordinary skill in the art, having the benefit of the present disclosure, will appreciate that alternative methods may be available without departing from the spirit and scope of the present disclosure.

### Exemplary Method 8.1

One or more embodiments of the present disclosure may comprise at least one of (i) performing the steps according to at least one of exemplary methods 3.1, 3.2, and 3.3 described hereinabove; (ii) adding 1 ml of bovine raw milk exosomes (5E13 EVs/ml); 0.5 ml of bovine raw milk ultracentrifugation peptide/protein fraction or 750 KDa TFF filtrate (100 KDa retentate) 50× concentrate; 1 ml of A-MSC exosomes (5E12 EVs/ml) at hyperbaric O₂ conditions; 1 ml of A-MSC secretome (MSC culture conditioned medium; TFF 750 KDa filtrate-3 KDa retentate) 50× concentrate at hyperbaric O₂ conditions; (iii) mixing at 35 rpm at 16° C. for 10 min; and (iv) adjusting to 20 g using base cream.

### Exemplary Embodiment 9

One or more embodiments of the present disclosure provide a composition for treating atopic dermatitis comprising bone marrow MSC exosomes.

By way of non-limiting example, one or more embodiments of the present disclosure comprise a vanishing base cream, such as Humco PENcream™; Bone Marrow MSC exosomes; Bone Marrow MSC secretome (MSC culture conditioned medium; TFF 750 KDa filtrate-3 KDa retentate); and any combination thereof.

In one or more embodiments, a composition comprises a vanishing base cream, such as Humco PENcream™; Bone Marrow MSC exosomes (1E13 EVs/ml); Bone Marrow MSC secretome (MSC culture conditioned medium; TFF 750 KDa filtrate-3 KDa retentate) 50× concentrate; and any combination thereof.

Exemplary methods for preparing the disclosed atopic dermatitis compositions are herein provided. One of ordinary skill in the art, having the benefit of the present disclosure, will appreciate that alternative methods may be available without departing from the spirit and scope of the present disclosure.

### Exemplary Method 9.1

One or more embodiments of the present disclosure may comprise at least one of (i) performing the steps according to at least one of exemplary methods 3.1, 3.2, and 3.3 described hereinabove; (ii) adding 1 ml of Bone Marrow MSC exosomes (1E13 EVs/ml); (iii) adding 1 ml of Bone Marrow MSC secretome (MSC culture conditioned medium; TFF 750 KDa filtrate-3 KDa retentate) 50× concentrate; (iv) mixing at 35 rpm in 16° C. for 10 min; and (v) Adjust to 50 g using base cream.

### Exemplary Embodiment 10

One or more embodiments of the present disclosure provide a lip cream composition comprising at least one of umbilical cord MSC exosomes and umbilical cord MSC secretome.

By way of non-limiting example, one or more embodiments comprise a highlighter balm; Umbilical cord MSC exosomes; Umbilical cord MSC secretome (MSC culture conditioned medium; TFF 750 KDa filtrate-3 KDa retentate); and any combination thereof.

One or more exemplary embodiments may comprise a highlighter balm (1445, for example); Umbilical cord MSC exosomes (5E12 EVs/ml); Umbilical cord MSC secretome (MSC culture conditioned medium; TFF 750 KDa filtrate-3 KDa retentate) 50× concentrate; and any combination thereof.

Exemplary methods for preparing the disclosed day/night lip cream compositions are herein provided. One of ordinary skill in the art, having the benefit of the present disclosure, will appreciate that alternative methods may be available without departing from the spirit and scope of the present disclosure.

### Exemplary Method 10.1

One or more embodiments of the present disclosure may comprise at least one of (i) performing the steps according to at least one of exemplary methods 3.1, 3.2, and 3.3 described hereinabove; (ii) adding 1 ml of Bone Marrow MSC exosomes (1E13 EVs/ml); (iii) adding 1 ml of Bone Marrow MSC secretome (MSC culture conditioned medium; TFF 750 KDa filtrate-3 KDa retentate) 50× concentrate; and (iv) adjust to 50 g using base cream.

In addition to the disclosed embodiments, one or more additional embodiments of the present disclosure may comprise preparing and/or providing one or more of the above-referenced compositions and applying said composition to the skin of an individual. Moreover, one or more embodiments may comprise preparing and/or providing one or more of the above-referenced compositions and instructing a person to apply the composition in accordance with a predefined treatment protocol, wherein the person applies the composition in accordance with said protocol.

Although particular embodiments have been disclosed herein in detail, this has been done for purposes of illustration only, and is not intended to be limiting with respect to the scope of any claims. In particular, it is contemplated by the inventors that various substitutions, alterations, and modifications may be made without departing from the spirit and scope of the embodiments described, including any variation in the amount and/or concentration of a component of the various embodiments.

### EXAMPLES

Microbial Challenge Test

A microbial challenge test was performed on an exemplary exosome-based anti-aging rejuvenating cream to evaluate the effectiveness of antimicrobial preservatives against microbial contamination. The exemplary embodiment comprised a base cream (Humco PENcream™) and A-MSC exosomes. The method employed was USP 42, Section 51, Antimicrobial Effectiveness Testing, as is known in the art.

Test organisms included Staphylococcus aureus ATCC#6538; Escherichia coli ATCC#8739; Pseudomonas aeruginosa ATCC#9027; Candida albicans ATCC#1023, and Aspergillus brasiliensis ATCC#16404.

Results

TABLE 2
Microbial Challenge Test Results (Log10 CFU/g or CFU/ml)

| Anti-aging | Staphylococcus | Escherichia | Pseudomonas | Candida | Aspergillus |
|---|---|---|---|---|---|
| cream | aureus | coli | aeruginosa | albicans | brasiliensis |
| Inoculum Level | 5.03 | 5.82 | 5.64 | 5.78 | 6.28 |
| Day 14 | 2.33 | <0.70 | <0.70 | 4.03 | 5.28 |
| Day 28 | <0.70 | <0.70 | <0.70 | 2.46 | 2.71 |

TABLE 3
Suitability of Counting Method Validation*

| | Product | Staphylococcus | Escherichia | Pseudomonas | Candida | Aspergillus |
|---|---|---|---|---|---|---|
| | Dilution | aureus | coli | aeruginosa | albicans | brasiliensis |
| Inert Control | N/A | 63.5 | 94 | 57 | 102 | 20 |
| Anti-aging | 10⁻¹ | 34 | 98 | 87 | 87.5 | 17.5 |

| | |
|---|---|
| cream | 10⁻² |
| | 10⁻³ |

*Suitability of the counting method is confirmed when recovery of the test organisms in the sample is at least 50% of that of the control. Test product dilutions past 10⁻¹ not reported where acceptable recovery is demonstrated on the 10⁻¹ dilution.

Acceptance Criteria

For category 2 products, the preservative is effective in the sample examined if a) the concentrations of viable bacteria demonstrate no less than a 2.0 log reduction from the initial count at 14 days and no increase from the day 14 count at 28 days; and b) the concentrations of viable yeast and molds demonstrate no increase from the initial calculated count at 14 and 28 days.

### CONCLUSION

The test material, Exosome-based anti-aging rejuvenating cream, conforms to the acceptance criteria for USP 42 <51> category 2 products.

Western Blot Characterization of Exosomes

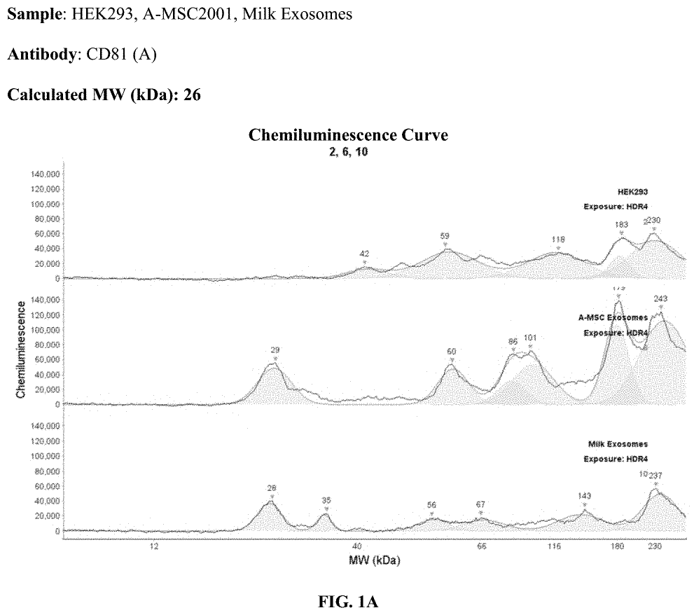

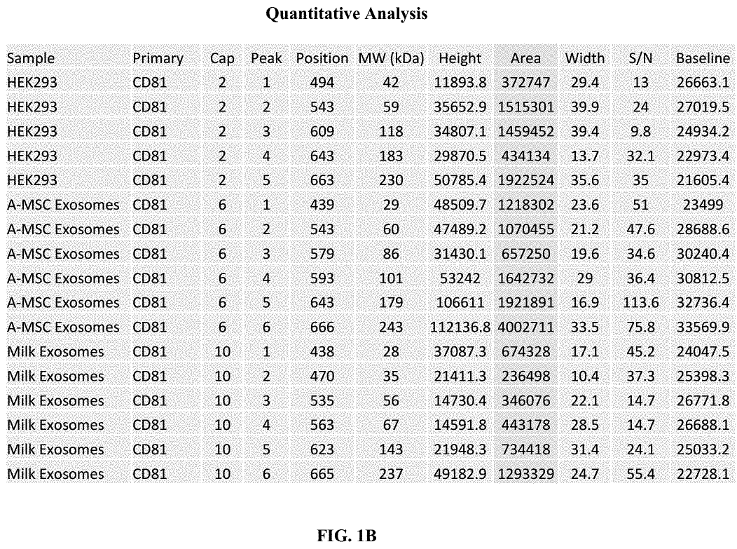

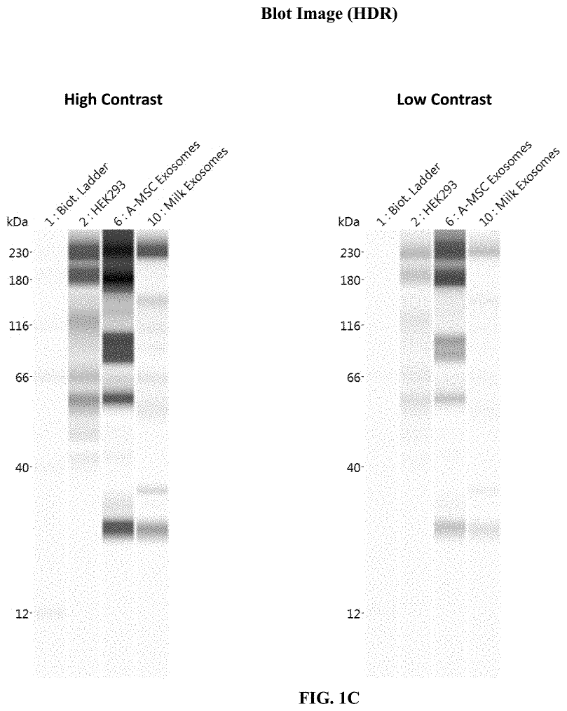

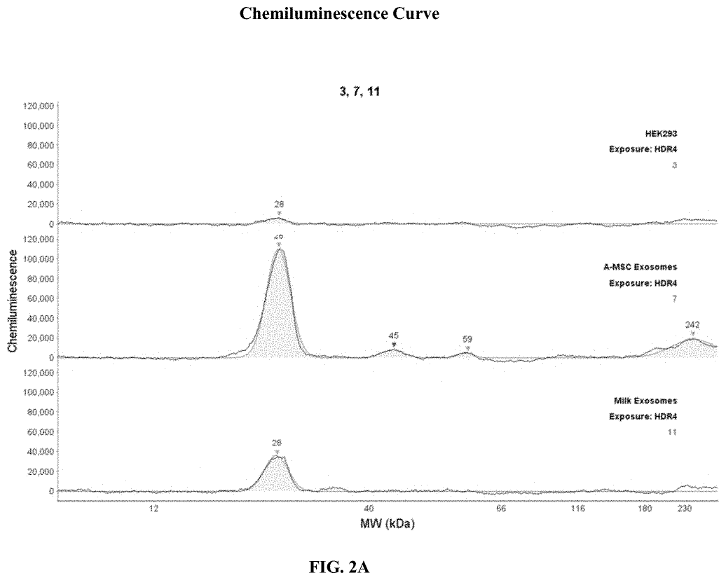

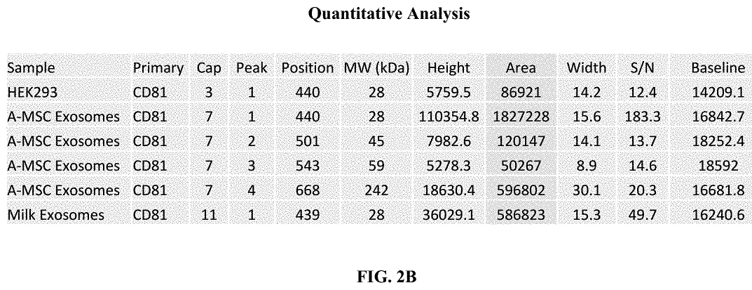

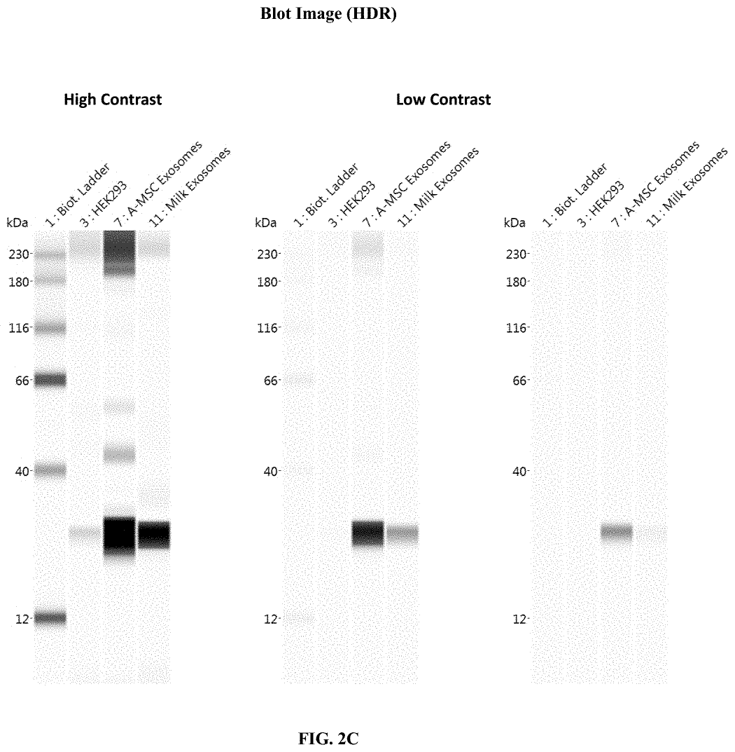

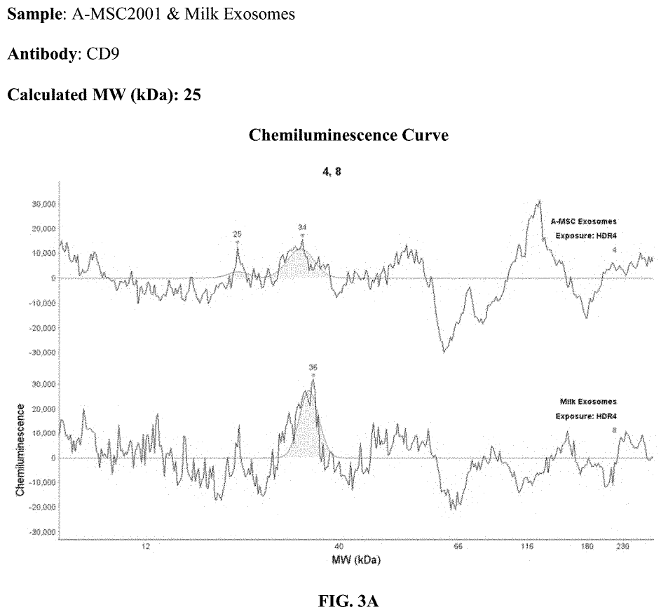

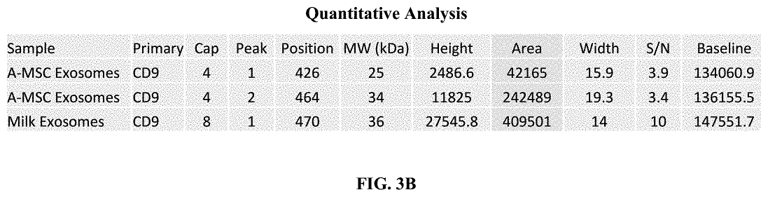

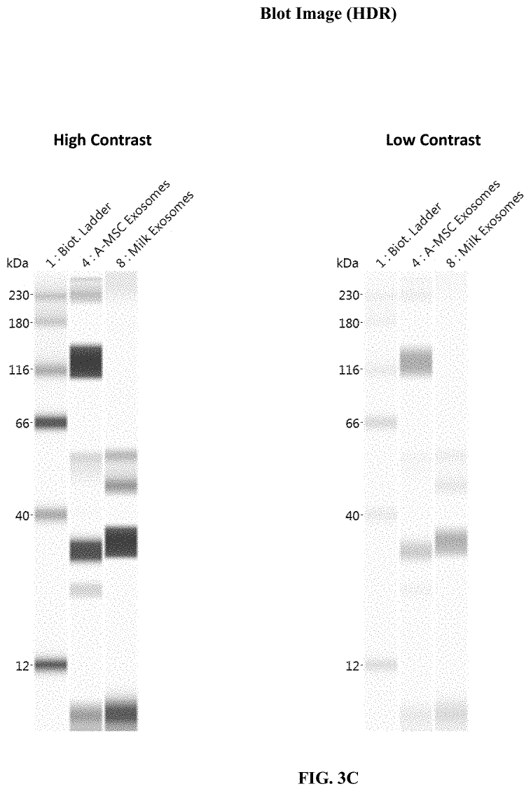

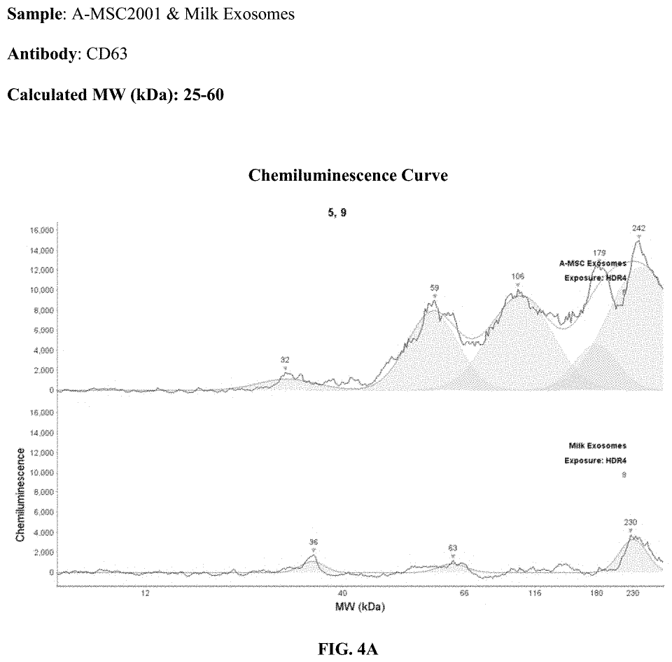

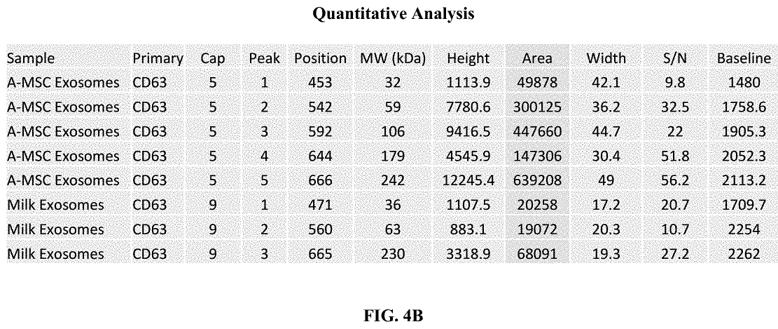

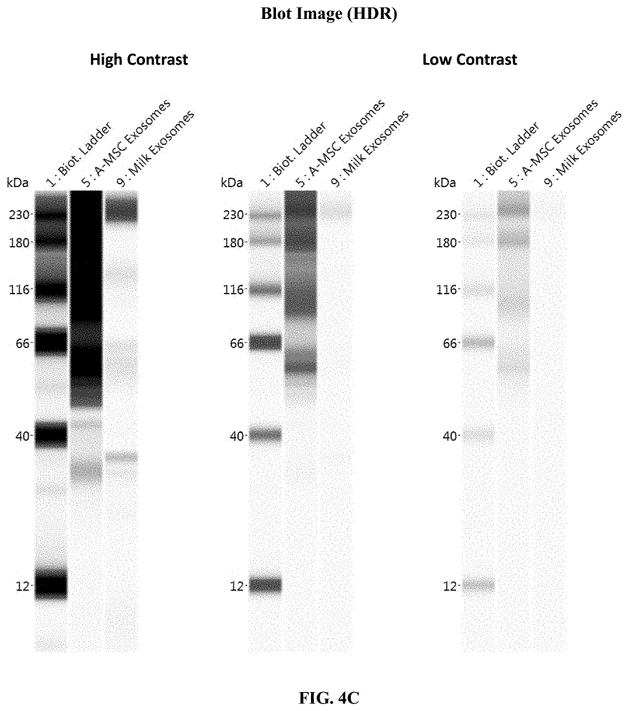

Exosomes were characterized by standard western blots using techniques known in the art, and as indicated in Table 4 below. The western blots demonstrate the presence of typical exosome biomarkers, thereby validating the exosome isolation techniques employed in connection with the embodiments disclosed herein. The results of this characterization are disclosed in FIGS. 1A-4C.

TABLE 4
Western Blot Criteria

| | |
|---|---|
| Sample #: | 3 |
| Primary Antibody: | 1. Anti-CD63 |
| | 2. Anti-CD9 |
| | 3. Anti-CD81 |
| Cell Lysate/Protein Type: | Exosomes |
| Secondary Antibody: | 1. Anti-Rabbit-HRP |
| | 2. Anti-Mouse-HRP |
| Calculated MW | CD63 25-60 kDa |
| | CD9 25 kDa |
| | CD81 26 kDa |
| Note: | Samples were loaded neat. CD81 (A) was |
| | used with previous experiments and CD81 |
| | (B) has recently been validated for |
| | Simple Wes. All previously used antibodies |
| | were used at 2X concentration to previous |
| | runs with EriVan Bio. |

Pyrogenic Materials

Lipopolysaccharide (LPS) content was measured using techniques known in the art.

LIPIDOMICS

Sample type: A-MSC Exosome

### Extraction Method: Bligh and Dyer

### Internal Standards: SPLASH+Lipidyzer

LC: Shimadzu LC system

### MS Instrument: Linear Ion Trap Quadrupole LC/MS/MS Mass Spectrometer

### Model: QTRAP 6500

### Company: AB SCIEX Instruments

### Scan Type: MRM (Scheduled)

### Polarity: Positive/Negative

Injection volume: 5 uL

### Unit: μM

LC Column: 1-Amide Column: XBridge Amide 3.5 μm, 4.6×150 mm column (Waters, Ireland)
Mobile phase A: Acetonitrile: Water (95:5) with 1 mM Ammonium acetate, pH=8.2
Mobile phase B: Acetonitrile: Water (50:50) with 1 mM Ammonium acetate, pH=8.2
Data explanation:
1—20 μL exosome was used for lipid extraction.
2—Lipids were quantified semi-quantitatively. Data were first normalized based on internal standards and concentrations were calculated based on a surrogate internal standard.
Lipopolysaccharides: Not detected

Lipid Content of A-MSC Exosomes

Lipid content was measured under the following criteria.

LIPIDOMICS

Sample type: A-MSC Exosome

### Extraction Method: Bligh and Dyer

### Internal Standards: SPLASH+Lipidyzer

LC: Shimadzu LC system

### MS Instrument: Linear Ion Trap Quadrupole LC/MS/MS Mass Spectrometer

### Model: QTRAP 6500

### Company: AB SCIEX Instruments

### Scan Type: MRM (Scheduled)

### Polarity: Positive/Negative

Injection volume: 5 uL

### Unit: μM

LC Column: 1-Amide Column: XBridge Amide 3.5 μm, 4.6×150 mm column (Waters, Ireland)
Mobile phase A: Acetonitrile: Water (95:5) with 1 mM Ammonium acetate, pH=8.2
Mobile phase B: Acetonitrile: Water (50:50) with 1 mM Ammonium acetate, pH=8.2
Data explanation:
1—20 μL exosome was used for lipid extraction.
2—Lipids were quantified semi-quantitatively. Data were first normalized based on internal standards and concentrations were calculated based on a surrogate internal standard.

TABLE 5
Lipid Content Results for A-MSC Exosomes.

| | Concentration |
|---|---|
| Lipid Species | (μM) |
| SM(14:0) + H | 0.07213 |
| SM(16:0) + H | 0.71884 |
| SM(18:0) + H | 0.04687 |
| SM(18:1) + H | 0.01309 |
| SM(20:0) + H | 0.01032 |
| SM(20:1) + H | 0.00299 |
| SM(22:0) + H | 0.05655 |
| SM(22:1) + H | 0.02795 |
| SM(24:0) + H | 0.11129 |
| SM(24:1) + H | 0.31208 |
| SM(26:0) + H | 0.00285 |
| SM(26:1) + H | 0.00609 |
| CE(24:0) + H | 1.91129 |
| CE(14:0) + H | 16.44606 |
| CE(18:3) + H | 3.70328 |
| CE(20:2) + H | 1.66904 |
| CE(22:0) + H | 4.06422 |
| CE(22:1) + H | 6.46284 |
| CER(14:0) + H | 0.62088 |
| CER(16:0) + H | 0.02152 |
| CER(22:0) + H | 0.01057 |
| CER(24:1) + H | 0.05315 |
| DCER(14:0) + H | 0.02842 |
| DCER(20:0) + H | 0.00891 |
| DCER(20:1) + H | 0.01992 |
| DCER(22:0) + H | 0.00335 |
| DCER(22:1) + H | 0.00128 |
| DCER(24:0) + H | 0.02728 |
| DCER(24:1) + H | 0.00584 |
| HCER(18:1) + H | 0.00028 |
| HCER(20:1) + H | 0.00038 |
| HCER(22:1) + H | 0.00033 |
| HCER(24:0) + H | 0.00015 |
| HCER(24:1) + H | 0.00050 |
| HCER(26:0) + H | 0.00007 |
| HCER(26:1) + H | 0.00011 |
| HCER(d18:0/20:0) + H | 0.00013 |
| TAG(44:2/FA16:1) + NH4 | 0.03816 |
| TAG(45:1/FA18:1) + NH4 | 0.02162 |
| TAG(47:2/FA18:1) + NH4 | 0.08057 |
| TAG(48:0/FA18:0) + NH4 | 0.06360 |
| TAG(48:2/FA18:2) + NH4 | 0.02968 |
| TAG(48:4/FA20:4) + NH4 | 0.05512 |
| TAG(49:1/FA16:0) + NH4 | 0.09150 |
| TAG(49:1/FA17:0) + NH4 | 0.04071 |
| TAG(49:2/FA18:1) + NH4 | 0.22205 |
| TAG(49:2/FA18:2) + NH4 | 0.09552 |
| TAG(49:3/FA18:2) + NH4 | 0.19554 |
| TAG(50:1/FA18:0) + NH4 | 0.08904 |
| TAG(50:1/FA18:1) + NH4 | 0.06458 |
| TAG(50:1/FA20:1) + NH4 | 0.05048 |
| TAG(50:2/FA18:1) + NH4 | 0.11208 |
| TAG(50:2/FA18:2) + NH4 | 0.06785 |
| TAG(50:3/FA18:2) + NH4 | 0.12883 |
| TAG(50:4/FA18:1) + NH4 | 0.06785 |
| TAG(50:4/FA18:3) + NH4 | 0.05863 |
| TAG(50:5/FA14:0) + NH4 | 0.06881 |
| TAG(50:5/FA18:2) + NH4 | 0.04411 |
| TAG(50:5/FA18:3) + NH4 | 0.02969 |
| TAG(50:5/FA20:5) + NH4 | 0.06785 |
| TAG(51:0/FA17:0) + NH4 | 0.07944 |
| TAG(51:1/FA16:0) + NH4 | 0.07615 |
| TAG(51:1/FA17:0) + NH4 | 0.79503 |
| TAG(51:1/FA18:0) + NH4 | 0.01620 |
| TAG(51:1/FA18:1) + NH4 | 0.05196 |
| TAG(51:2/FA16:0) + NH4 | 0.27344 |
| TAG(51:2/FA16:1) + NH4 | 0.02286 |
| TAG(51:2/FA18:1) + NH4 | 0.20526 |
| TAG(51:2/FA18:2) + NH4 | 0.03529 |
| TAG(51:3/FA16:1) + NH4 | 0.02544 |
| TAG(51:4/FA18:3) + NH4 | 0.02400 |
| TAG(52:1/FA20:0) + NH4 | 0.07916 |
| TAG(52:2/FA16:0) + NH4 | 0.03179 |
| TAG(52:2/FA18:1) + NH4 | 0.05192 |
| TAG(52:2/FA18:2) + NH4 | 0.00666 |
| TAG(52:3/FA14:0) + NH4 | 0.11646 |
| TAG(52:3/FA18:1) + NH4 | 0.07688 |
| TAG(52:3/FA18:2) + NH4 | 0.05766 |
| TAG(52:3/FA20:1) + NH4 | 0.03089 |
| TAG(52:3/FA20:2) + NH4 | 0.04207 |
| TAG(52:4/FA16:1) + NH4 | 0.02831 |
| TAG(52:4/FA18:2) + NH4 | 0.04908 |
| TAG(52:4/FA20:4) + NH4 | 0.03398 |
| TAG(52:5/FA20:5) + NH4 | 0.02161 |
| TAG(52:6/FA20:5) + NH4 | 0.01179 |
| TAG(52:8/FA18:2) + NH4 | 0.01966 |
| TAG(53:1/FA17:0) + NH4 | 0.33342 |
| TAG(53:2/FA17:0) + NH4 | 0.44098 |
| TAG(53:2/FA18:1) + NH4 | 0.08822 |
| TAG(53:3/FA17:0) + NH4 | 0.55277 |
| TAG(53:4/FA17:0) + NH4 | 0.16591 |
| TAG(53:4/FA18:2) + NH4 | 0.15979 |
| TAG(53:6/FA20:4) + NH4 | 0.01178 |
| TAG(54:1/FA18:1) + NH4 | 0.08254 |
| TAG(54:2/FA18:1) + NH4 | 0.04323 |
| TAG(54:2/FA20:2) + NH4 | 0.14525 |
| TAG(54:3/FA18:0) + NH4 | 0.01296 |
| TAG(54:3/FA18:1) + NH4 | 0.09068 |
| TAG(54:4/FA18:1) + NH4 | 0.03851 |
| TAG(54:5/FA18:1) + NH4 | 0.01418 |
| TAG(54:5/FA18:2) + NH4 | 0.01032 |
| TAG(54:6/FA18:1) + NH4 | 0.00838 |
| TAG(54:6/FA18:2) + NH4 | 0.01640 |
| TAG(54:6/FA20:4) + NH4 | 0.01032 |
| TAG(54:6/FA22:6) + NH4 | 0.01592 |
| TAG(54:7/FA18:3) + NH4 | 0.00789 |
| TAG(54:7/FA22:6) + NH4 | 0.02672 |
| TAG(54:8/FA18:2) + NH4 | 0.00972 |
| TAG(55:2/FA18:1) + NH4 | 0.00486 |
| TAG(55:5/FA18:1) + NH4 | 0.02837 |
| TAG(56:3/FA18:1) + NH4 | 0.02282 |
| TAG(56:4/FA20:1) + NH4 | 0.00208 |
| TAG(56:4/FA20:2) + NH4 | 0.00092 |
| TAG(56:6/FA18:0) + NH4 | 0.00424 |
| TAG(56:6/FA22:5) + NH4 | 0.00318 |
| TAG(56:7/FA18:2) + NH4 | 0.00393 |
| TAG(58:6/FA20:4) + NH4 | 0.00092 |
| TAG(58:6/FA22:5) + NH4 | 0.00162 |
| TAG(60:11/FA22:6) + NH4 | 0.00190 |
| DAG(14:0/14:0) + NH4 | 1.07354 |
| DAG(14:0/16:1) + NH4 | 0.35656 |
| DAG(16:0/16:0) + NH4 | 0.44489 |
| DAG(16:0/16:1) + NH4 | 0.20687 |
| DAG(14:0/18:1) + NH4 | 11.56397 |
| DAG(16:1/16:1) + NH4 | 0.32172 |
| DAG(14:0/18:2) + NH4 | 11.76252 |
| DAG(16:1/18:0) + NH4 | 0.86065 |
| DAG(16:0/18:1) + NH4 | 7.43409 |
| DAG(16:1/18:1) + NH4 | 25.01781 |
| DAG(16:1/18:2) + NH4 | 0.81119 |
| DAG(16:0/18:3) + NH4 | 0.48630 |
| DAG(16:1/18:3) + NH4 | 0.30584 |
| DAG(14:0/20:4) + NH4 | 0.48265 |
| DAG(16:1/20:0) + NH4 | 0.54642 |
| DAG(18:0/18:1) + NH4 | 0.53764 |
| DAG(18:1/18:1) + NH4 | 1.56569 |
| DAG(18:0/18:2) + NH4 | 1.18960 |
| DAG(18:1/18:2) + NH4 | 0.28234 |
| DAG(18:0/18:3) + NH4 | 0.40922 |
| DAG(16:1/20:2) + NH4 | 0.45044 |
| DAG(16:0/20:3) + NH4 | 0.12535 |
| DAG(16:0/20:4) + NH4 | 0.12352 |
| DAG(16:1/20:4) + NH4 | 0.35612 |
| DAG(16:0/20:5) + NH4 | 0.06973 |
| DAG(14:0/22:6) + NH4 | 0.12015 |
| DAG(18:2/20:5) + NH4 | 0.48798 |
| DAG(18:1/22:4) + NH4 | 0.31248 |
| DAG(18:0/22:6) + NH4 | 12.80632 |
| MAG(16:0) + NH4 | 1.77107 |
| MAG(16:1) + NH4 | 2.15081 |
| MAG(18:0) + NH4 | 16.11039 |
| MAG(18:1) + NH4 | 17.46537 |
| MAG(18:2) + NH4 | 2.54008 |
| MAG(20:0) + NH4 | 1.88197 |
| MAG(20:1) + NH4 | 1.94088 |
| MAG(20:2) + NH4 | 1.77442 |
| MAG(20:3) + NH4 | 2.21464 |
| MAG(20:4) + NH4 | 1.43079 |
| MAG(22:0) + NH4 | 0.80881 |
| MAG(22:1) + NH4 | 0.90217 |
| MAG(22:2) + NH4 | 1.25383 |
| MAG(22:3) + NH4 | 1.40984 |
| MAG(22:4) + NH4 | 0.60677 |
| MAG(22:5) + NH4 | 1.50153 |
| MAG(22:6) + NH4 | 1.26461 |
| LPC(16:0) + AcO | 0.01471 |
| LPC(16:1) + AcO | 0.00329 |
| LPC(18:0) + AcO | 0.02612 |
| LPC(18:1) + AcO | 0.01745 |
| LPC(18:2) + AcO | 0.01966 |
| LPC(20:0) + AcO | 0.21392 |
| LPC(20:4) + AcO | 0.03161 |
| LPC(20:5) + AcO | 0.00890 |
| PC(16:0/20:4) + AcO | 0.31803 |
| PC(16:0/22:6) + AcO | 0.11441 |
| PC(18:0/18:0) + AcO | 0.46047 |
| PC(18:0/20:0) + AcO | 1.14246 |
| PC(18:0/20:3) + AcO | 0.16399 |
| PC(18:0/20:4) + AcO | 0.49047 |
| PC(18:0/22:5) + AcO | 0.12330 |
| PC(18:0/22:6) + AcO | 0.19950 |
| PC(18:1/20:4) + AcO | 0.13123 |
| PC(18:1/22:6) + AcO | 0.04596 |
| PC(20:0/20:4) + AcO | 0.04276 |
| PC(20:0/22:6) + AcO | 0.02643 |
| LPE(16:0) − H | 0.00173 |
| LPE(16:1) − H | 0.00026 |
| LPE(18:0) − H | 0.00104 |
| LPE(18:1) − H | 0.00119 |
| LPE(18:2) − H | 0.00261 |
| LPE(18:3) − H | 0.00027 |
| LPE(20:0) − H | 0.00006 |
| LPE(20:1) − H | 0.00015 |
| LPE(20:2) − H | 0.00016 |
| LPE(20:3) − H | 0.00228 |
| LPE(20:4) − H | 0.00486 |
| LPE(20:5) − H | 0.00163 |
| LPE(22:4) − H | 0.00012 |
| PE(14:0/18:1) − H | 0.00171 |
| PE(16:0/18:1) − H | 0.03411 |
| PE(16:0/18:2) − H | 0.00181 |
| PE(16:0/20:1) − H | 0.00077 |
| PE(16:0/20:2) − H | 0.00127 |
| PE(16:0/20:3) − H | 0.00331 |
| PE(16:0/20:4) − H | 0.00304 |
| PE(16:0/22:4) − H | 0.00499 |
| PE(16:0/22:5) − H | 0.00284 |
| PE(16:0/22:6) − H | 0.00067 |
| PE(18:0/16:0) − H | 0.00136 |
| PE(18:0/16:1) − H | 0.00276 |
| PE(18:0/18:0) − H | 0.00385 |
| PE(18:0/18:1) − H | 0.08735 |
| PE(18:0/18:2) − H | 0.00751 |
| PE(18:0/20:1) − H | 0.00173 |
| PE(18:0/20:2) − H | 0.00556 |
| PE(18:0/20:3) − H | 0.02056 |
| PE(18:0/20:4) − H | 0.02382 |
| PE(18:0/22:4) − H | 0.01556 |
| PE(18:0/22:5) − H | 0.00432 |
| PE(18:1/16:1) − H | 0.00354 |
| PE(18:1/18:1) − H | 0.13849 |
| PE(18:1/18:2) − H | 0.00612 |
| PE(18:1/20:1) − H | 0.00389 |
| PE(18:1/20:2) − H | 0.00520 |
| PE(18:1/20:3) − H | 0.01514 |
| PE(18:1/20:4) − H | 0.01362 |
| PE(18:1/22:4) − H | 0.00796 |
| PE(18:1/22:5) − H | 0.00813 |
| PE(O-16:0/16:0) − H | 0.00266 |
| PE(O-16:0/16:1) − H | 0.00200 |
| PE(O-16:0/18:1) − H | 0.00182 |
| PE(O-16:0/18:3) − H | 0.00095 |
| PE(O-16:0/20:4) − H | 0.00868 |
| PE(O-16:0/20:5) − H | 0.00380 |
| PE(P-16:0/16:0) − H | 0.00418 |
| PE(P-16:0/16:1) − H | 0.00458 |
| PE(P-16:0/18:0) − H | 0.00122 |
| PE(P-16:0/18:1) − H | 0.04969 |
| PE(P-16:0/18:2) − H | 0.00365 |
| PE(P-16:0/18:3) − H | 0.00087 |
| PE(P-16:0/20:1) − H | 0.00917 |
| PE(P-16:0/20:2) − H | 0.01433 |
| PE(P-16:0/20:3) − H | 0.04122 |
| PE(P-16:0/20:4) − H | 0.04492 |
| PE(P-16:0/22:4) − H | 0.05504 |
| PE(P-16:0/22:5) − H | 0.39469 |
| PE(P-16:1/18:1) − H | 0.00062 |
| PE(P-18:0/16:0) − H | 0.00670 |
| PE(P-18:0/16:1) − H | 0.00416 |
| PE(P-18:0/18:0) − H | 0.00061 |
| PE(P-18:0/18:1) − H | 0.01650 |
| PE(P-18:0/18:2) − H | 0.00234 |
| PE(P-18:0/20:1) − H | 0.00250 |
| PE(P-18:0/20:2) − H | 0.00315 |
| PE(P-18:0/20:3) − H | 0.01315 |
| PE(P-18:0/20:4) − H | 0.04276 |
| PE(P-18:0/22:4) − H | 0.02263 |
| PE(P-18:0/22:5) − H | 0.00620 |
| PE(P-18:0/22:6) − H | 0.00381 |
| PE(P-18:1/16:0) − H | 0.03127 |
| PE(P-18:1/16:1) − H | 0.00643 |
| PE(P-18:1/18:0) − H | 0.00113 |
| PE(P-18:1/18:1) − H | 0.03018 |
| PE(P-18:1/18:2) − H | 0.00297 |
| PE(P-18:1/20:1) − H | 0.00317 |
| PE(P-18:1/20:2) − H | 0.00399 |
| PE(P-18:1/20:3) − H | 0.02934 |
| PE(P-18:1/20:4) − H | 0.08198 |
| PE(P-18:1/22:4) − H | 0.02682 |
| PE(P-18:1/22:5) − H | 0.02253 |
| PE(P-18:1/22:6) − H | 0.00262 |
| PG(18:0/18:1) − H | 0.00027 |
| PG(18:0/18:2) − H | 0.00004 |
| PG(18:1/18:2) − H | 0.00004 |
| PG(18:1/20:4) − H | 0.00003 |
| PG(20:0/18:1) − H | 0.00069 |
| PG(20:0/18:2) − H | 0.00062 |
| PG(20:0/18:3) − H | 0.00013 |
| PG(20:0/20:3) − H | 0.00015 |
| PG(20:0/20:4) − H | 0.00047 |
| PG(20:0/20:5) − H | 0.00007 |
| PG(20:0/22:5) − H | 0.00009 |
| PG(20:0/22:6) − H | 0.00017 |
| PI(18:0/20:3) − H | 0.00585 |
| PI(18:0/20:4) − H | 0.00599 |
| PS(16:0/20:3) − H | 0.00020 |
| PS(16:0/22:4) − H | 0.00022 |
| PS(16:0/22:6) − H | 0.00004 |
| PS(18:0/20:4) − H | 0.00011 |
| PS(18:1/20:3) − H | 0.00028 |
| PS(18:1/20:4) − H | 0.00056 |
| PS(18:1/22:4) − H | 0.00023 |
| PS(18:1/22:5) − H | 0.00005 |
| PS(18:2/20:3) − H | 0.00030 |
| PS(18:2/20:4) − H | 0.00050 |
| PS(18:2/22:4) − H | 0.00038 |

Cell Proliferation and Cytotoxicity Assays

MTT Assay

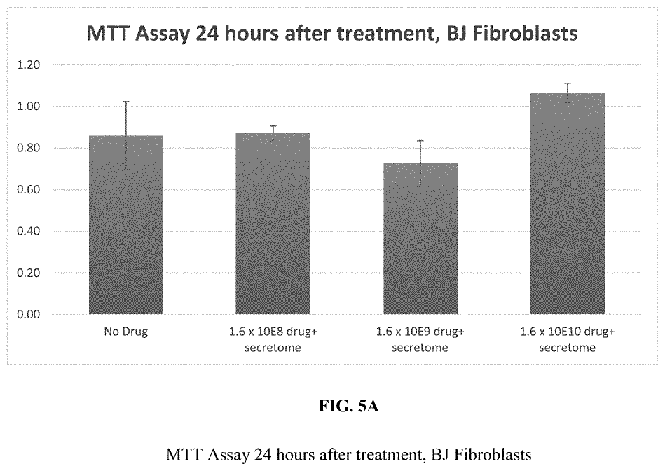

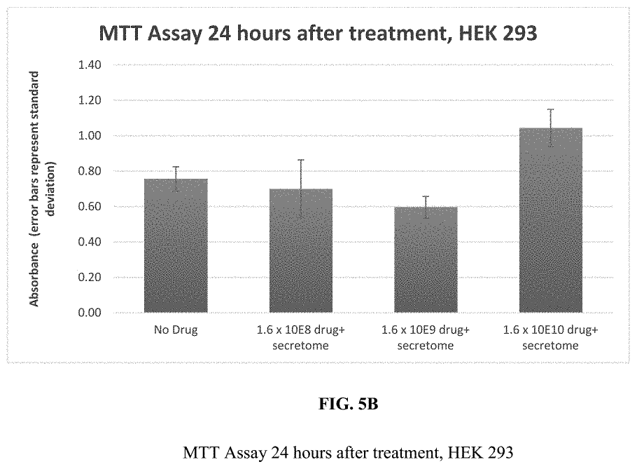

The BJ Fibroblasts were seeded at 1E4 cells in a 96-well plate in EMEM and 10% FBS in a final volume of 100 ul/well culture medium in a humidified atmosphere (37° C., 5% CO₂) and penicillin and streptomycin. The HEK 293 cells were seeded at 1E4 cells in a 96-well plate in DMEM and 10% FBS in a final volume of 100 ul/well culture medium in a humidified atmosphere (37° C., 5% CO₂). After two days of culture, A-MSC exosomes and secretome was added to the wells in different concentrations. After different time points, 10 ul of the MTT dye (Sigma Aldrich) was added to each well, and the plate was incubated at 37° C. for 4 h in a humidified atmosphere and under 5% CO₂. The media content of each well was carefully removed without disturbing cells. Then, 100 μl of DMSO was added to each well and mixed by pipetting up and down. The plate was incubate at 37° C. for 15 minutes. After gentle mixing on a shaker for 1 min, the absorbance of the samples against a background control as blank was measured using a microplate reader (BioMate 5, Thermo Spectronic) at 570 nm. The results are depicted in FIGS. 5A-5B.

WST-1 Assays

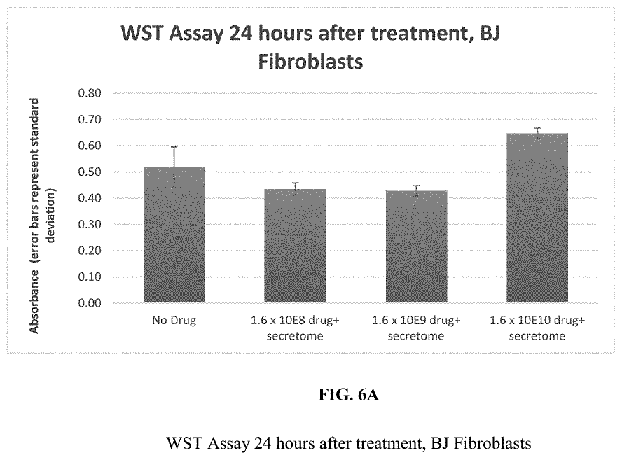

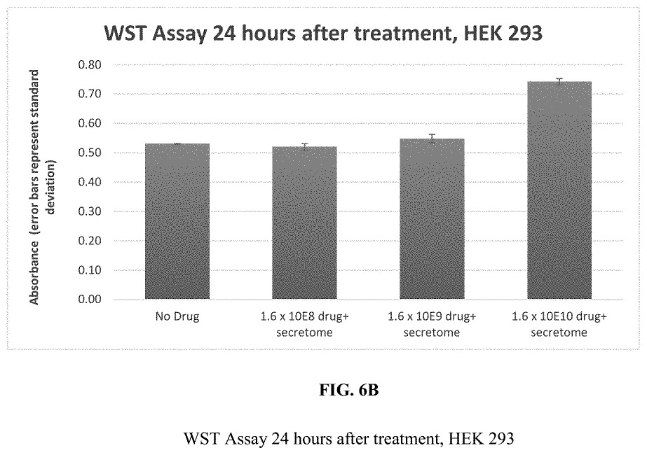

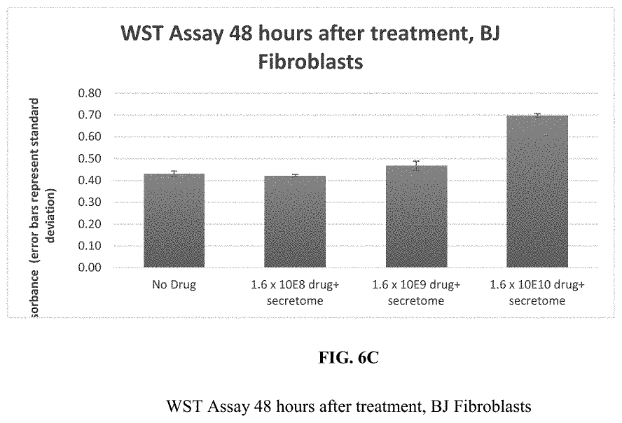

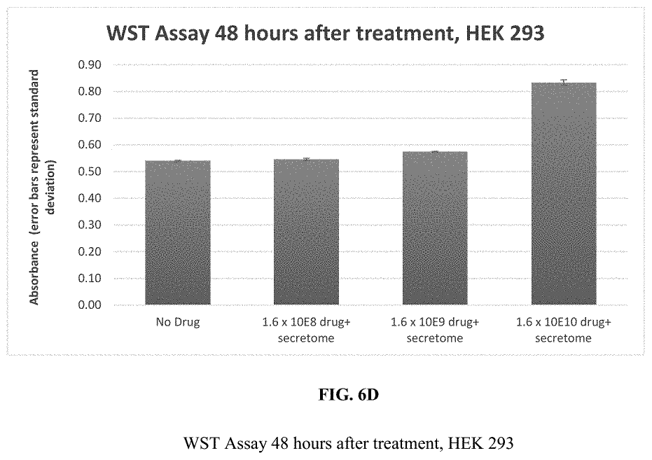

The BJ Fibroblasts were seeded at 1E4 cells in a 96-well plate in EMEM and 10% FBS in a final volume of 100 ul/well culture medium in a humidified atmosphere (37° C., 5% CO₂) and penicillin and streptomycin. The HEK 293 cells were seeded at 1E4 cells in a 96-well plate in DMEM and 10% FBS in a final volume of 100 ul/well culture medium in a humidified atmosphere (37° C., 5% CO₂). After two days of culture, A-MSC exosomes and secretome was added to the wells in different concentrations. After different time points, 10 ul of the WST-1 reagent (Roche Applied Science) was added to each well, and the plate was incubated at 37° C. for 2 h in a humidified atmosphere and under 5% CO₂. After gentle mixing on a shaker for 1 min, the absorbance of the samples against a background control as blank was measured using a microplate reader (BioMate 5, Thermo Spectronic) at 450 nm. The results are depicted in FIGS. 6A-6D.

The results show that different concentration of exosomes and secretome have different effects on the BJ fibroblasts and HEK 293 cells. A concentration dependent cell proliferation and viability effect is observed in the BJ fibroblasts and HEK 293 cells. The lower concentrations of exosomes and secretome did not produce significant improvement in cell proliferation in contrast to E10 concentration. Similar trend is observed in both cell types. However, MTT and WST-1 assays resulted in similar viability profiles. The WST-1 assay shows that the enzymatic activity of the BJ fibroblasts improves by 24.9% in 24 hours while the effect is 62.1% in 48 hours. Similar pattern is observed in HEK 293 cells with 40% after 24 hours against 54.5% in 48 hours.

Wound Healing Effect (Scratch Assay)

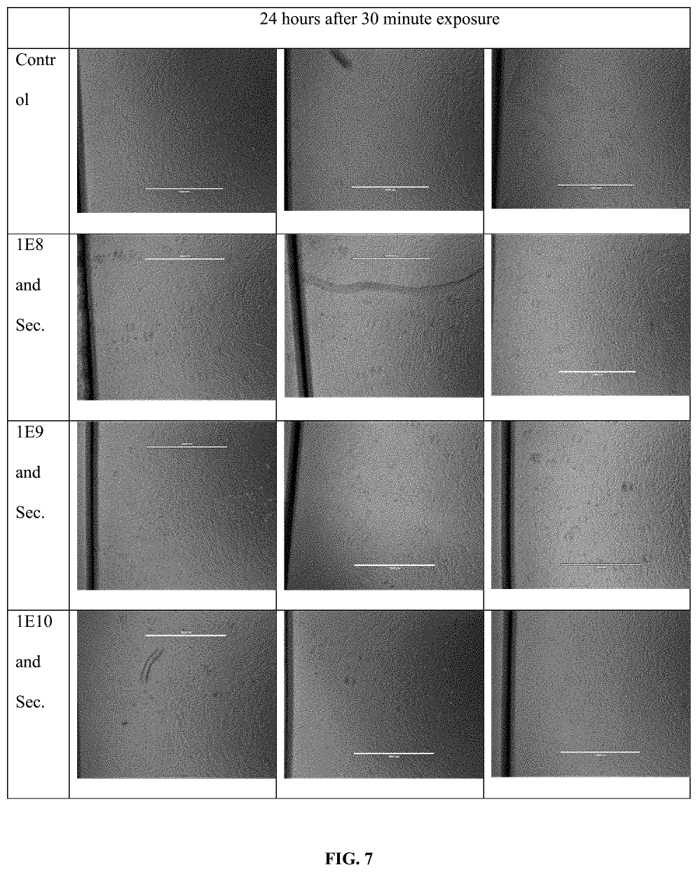

The BJ Fibroblasts were seeded at 5E4 cells in a 24-well plate in EMEM and 10% FBS in a final volume of 1 ml/well culture medium and penicillin and streptomycin in a humidified atmosphere (37° C., 5% CO₂). After two days of culture, the plates were exposed to UV light at 365 nm for 10 or 30 minutes using a mini transilluminator machine (BioRad). Then, A-MSC exosomes and secretome was added to the wells in different concentrations. Then a straight scratch was made in each well using a sterile 200 μl pipette tip. At different time points, images of wound scratch were taken under a microscope (Fluorescent, Inverted EVOS FL Life Technologies (Evos)). And scratch areas were analyzed by using Image J (NIH, USA). The results are depicted in FIG. 7.

Compared to control cells, the migration of fibroblasts into scratched areas of monolayers in the presence of 1E8, 1E9 and 1E10 A-MSC exosomes and 50 ul secretome (40× concentrated) increased by 21.8%, 20.8% and 62.1% after 24 h, respectively.

What is claimed is:

1. A topical composition for treating a subject, the composition comprising:
a base cream; and
an exosome.

2. The composition of claim 1, wherein the exosome comprises a milk exosome.

3. The composition of claim 2, further comprising at least one of minoxidil, hydroxypropyl-β-cyclodextrin (HPbCD), and any combination thereof.

4. The composition of claim 2, further comprising at least one of hydroquinone, vitamin A, vitamin B3, arbutin, kojic acid, viola tricolor ethanolic extract, and any combination thereof.

5. The composition of claim 4, wherein the milk exosomes comprise bovine raw milk exosomes.

6. The composition of claim 4, further comprising at least one of an MSC exosome and an MSC secretome.

7. The composition of claim 2, further comprising at least one of Vitamin A, Vitamin B3, Vitamin E, hydrolyzed collagen, hyaluronic acid, jojoba oil, allantoin, fibronectin, and any combination thereof.

8. The composition of claim 7, wherein the milk exosomes comprise bovine raw milk exosomes.

9. The composition of claim 7, further comprising at least one of an MSC exosome and an MSC Secretome.

10. The composition of claim 9, wherein the MSC exosome comprises an A-MSC exosome.

11. The composition of claim 2, further comprising at least one of Vitamin C, ferulic acid, hyaluronic acid, retinol, and any combination thereof.

12. The composition of claim 11, further comprising at least one of Vitamin A, Vitamin B3; Vitamin E, magnesium ascorbyl phosphate (MAP), ascorbyl 6 palmitate, disodium isostearyl 2-0 L-ascorbyl phosphate (VCP-IS-Na), tetraisopalmitoyl ascorbic acid (TIPA), ascorbic acid sulfate; coenzyme Q10 (ubiquinone), lactic acid, hydrolyzed collagen, hyaluronic acid, and any combination thereof.

13. The composition of claim 11, wherein the milk exosomes comprise bovine raw milk exosomes.

14. The composition of claim 11, further comprising at least one of an MSC exosome and an MSC secretome.

15. The composition of claim 2, further comprising at least one of an umbilical cord MSC exosome and an umbilical cord MSC secretome.

16. The composition of claim 15, further comprising at least one of Vitamin A, Vitamin B3, Vitamin E, bovine raw milk exosomes, and any combination thereof.

17. The composition of claim 2, further comprising at least one of a retinol and an A-MSC exosome.

18. The composition of claim 17, wherein the A-MSC exosome was generated under hyperbaric O₂ conditions.

19. A topical composition for treating a subject, the composition comprising at least one of an A-MSC exosome and an A-MSC secretome.

20. The composition of claim 19, further comprising a milk exosome.

21. The composition of claim 19, wherein at least one of the A-MSC exosome and the A-MSC secretome was generated under hyperbaric O₂ conditions.

22. The composition of claim 21, further comprising at least one of platelet-rich plasma (PRP), induced pluripotent stem cells (iPCs), Vitamin A, Vitamin E, PRP isolated exosomes, iPCs isolated exosomes, and any combination thereof.

23. The composition of claim 19, further comprising at least one of an angiogenic factor and Nrf2.

24. The composition of claim 23, further comprising at least one of Vitamin A and Vitamin E.

25. A composition for treating a subject, the composition comprising at least one of a bone marrow MSC exosome and a bone marrow MSC secretome.

26. A composition for treating a subject, the composition comprising a highlighter balm, an umbilical cord MSC exosome, an umbilical cord MSC secretome, and any combination thereof.
